SEQUENCE LISTING (8 sequences)

SEQ ID NO: 1 - DNA, 37 bp, Artificial Sequence
  Synthetic primer
ggcctcgagagatctccaccatgagcacagaaagcat

SEQ ID NO: 2 - DNA, 41 bp, Artificial Sequence
  Synthetic primer
gagggaggccatttgggaacttctcatccctttggggaccg

SEQ ID NO: 3 - DNA, 41 bp, Artificial Sequence
  Synthetic Primer
cggtccccaaagggatgagaagttcccaaatggcctccctc

SEQ ID NO: 4 - DNA, 42 bp, Artificial Sequence
  Synthetic primer
ctcgaattttgagaagatgatctgagtgtgagggtctgggcc

SEQ ID NO: 5 - DNA, 42 bp, Artificial Sequence
  Synthetic primer
ggcccagaccctcacactcagatcatcttctcaaaattcgag

SEQ ID NO: 6 - DNA, 39 bp, Artificial Sequence
  Synthetic primer
ttgcggatccggtacctcacagagcaatgactccaaagt

SEQ ID NO: 7 - DNA, 97 bp, Artificial Sequence
  Synthetic primer
ggccctcgagagatctctcaccatggagtttgggctgagctggctttttcttgtggctattttaaaaggtgtccagtgtctcagatcatcttctcaa

SEQ ID NO: 8 - DNA, 100 bp, Artificial Sequence
  Synthetic primer
ggccctcgagagatctctcaccatgagggtccccgctcagctcctggggctcctgctgctctggctcccaggtgcacgatgtctcagatcatcttctcaa

(19) United States
(12) Patent Application Publication
(10) Pub. No.: US 2002/0137670 A1
(43) Pub. Date: Sep. 26, 2002

(54) Transferrin polycation/DNA complexes for the systemic treatment of tumor diseases with cytotoxic proteins

(75) Inventors: Ralf Kircheis, Wien (AT); Ernst Wagner, Langenzersdorf (AT); Lionel Wightman, Wien (AT); Elinborg Ostermann, Wien (AT)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Boehringer Ingelheim International GmbH.

(21) Appl. No.: 09/969,139

(22) Filed: Oct. 3, 2001

(30) Foreign Application Priority Data
Oct. 4, 2000 (DE) 100 49 010.7

(51) Int. Cl. A61K 48/00; A61K 31/704; A61K 31/7072; A61K 31/475; C07K 14/79

(52) U.S. Cl. 514/6; 514/44; 530/395; 514/34; 514/50; 514/283; 514/449

(57) ABSTRACT

Complex of DNA, containing one or more DNA molecules coding for one or more therapeutically active proteins with a cytotoxic activity, and a polycation conjugated with transferrin with a zeta potential of ≦+15 mV. The complex, in which a high transferrin content screens the positive charge, produces a targeted transportation of the therapeutic DNA to the tumors in the systemic treatment of tumor diseases.

The present invention relates to the systemic gene therapy of tumour diseases.

The efficient killing off of tumour cells and the destruction of the living conditions of the tumour are the objective of all existing antitumour therapies. The most important condition of a therapeutically effective therapy is maximum possible, best of all total, damage to the tumour tissue, with the least possible damage to or influence on normal tissue.

With the exception of the surgical removal of the tumour the approaches to cancer therapy which currently exist, such as radiotherapy and chemotherapy, are mainly limited in their usefulness and efficiency by their toxicity to normal tissue or to the body as a whole.

The need to cause as little damage as possible or to have the least possible effect on the normal tissue or on the body as a whole whilst doing maximum damage to the tumour tissue also applies to the therapeutic use of biologically active substances, e.g. in the therapeutic use of biologically highly active mediators such as cytokines.

Of the known cytokines, tumour necrosis factor TNFα, and the closely related cytokines tumour necrosis factor-β (lymphotoxin), interleukin-1 (IL-1) and interleukin-6 are characterised by a particularly potent biological activity which is demonstrated on the one hand by a powerful activity even in a very low dosage range (pg-ng range) (Hohmann et al., 1990; Kramer et al., 1986), and by an extremely broad spectrum of activity on a multiplicity of target cells (Beutler and Cerami, 1986; Bendtzen, 1988).

TNF-α is a protein produced mainly by activated monocytes and macrophages under certain stress situations (Männel et al., 1980). TNF-α was originally discovered because of its particular quality of inducing haemorrhagic necrosis in tumours (Carswell et al., 1975; Old, 1985). The haemorrhagic necrosis can be attributed primarily to the damage and destruction of the blood vessels supplying the tumour, followed by coagulopathies which cause an interruption to the blood supply to the tumour and eventually lead to tumour necrosis (Old, 1985; Van de Wiel et al., 1989).

The damage to the blood vessels starts with activation of and, at higher doses of TNF-α, damage to the endothelial cells (Pober, 1988; Watanabe et al., 1988; Anderson et al., 1994). TNF-α induces a reduced secretion of thrombomodulin and an increased secretion of plasminogen activator inhibitor. This disrupts the clotting and fibrinolytic system, resulting in damage to the bloodflow (Van de Wiel et al., 1989; Anderson et al., 1994). This is further intensified by the vasodilatory effect of the prostaglandins and other mediators released (such as e.g. PAF) (Bachwich et al., 1986; Quinn and Slotman, 1999). In parallel with the haemostasis and coagulopathies there is an increase in the permeability of the capillaries and the leakage of fluid and macromolecules into the tissues (known as “capillary leakage syndrome”) (Old, 1985; Van de Wiel et al., 1989; Edwards et al., 1992; Ferrero et al., 1996; Renard et al., 1994; 1995).

Another component of the antitumour activity of TNF-α is the activation of inflammatory cells (such as macrophages, granulocytes) and immune cells such as T-cells, and B-lymphocytes. Macrophages are stimulated by TNF-α to increased cytotoxicity and to release IL-1, prostaglandins, M-CSF, GM-CSF and other mediators (Bachwich et al., 1986). Neutrophilic granulocytes are activated by TNF-α to increased phagocytosis and a greater release of lysozymes and oxygen radicals (Klebanoff et al., 1986; Yi and Ulich, 1992). TNF-α also activates T-lymphocytes to increase expression of IL-2 and TNF-α receptors, HLA-DR antigens and to release interferon-γ (Scheurich et al., 1987).

TNF-α also brings about an increased adhesion of inflammatory and immune cells. The chemotactic activity of TNF-α or of the mediators induced by it (such as IL-8), and the stimulation of the expression of a number of adhesion molecules (CD11, ELAM; ICAM) and of HLA antigens plays an important part (Collins et al., 1986; Renard et al., 1994; Westerman et al., 1999).

TNF-α also exhibits a direct cytotoxic effect on various tumour cell lines (Sugarman et al., 1985, Kircheis et al. 1992a).

The antitumour activity has been demonstrated predominantly in animal trials. After the injection of TNF-α marked haemorrhagic tumour necroses were shown in various transplanted tumours in mouse models (Old, 1985; Van de Wiel et al., 1989; Kircheis et al., 1992a). However, marked therapeutic effects were only visible after the administration of high doses of TNF-α, while doses that were too small had hardly any effect (Van de Viel et al., 1989; Kircheis et al., 1992a). It soon became apparent that the high doses of TNF-α required for therapeutic effects were accompanied by marked systemic toxicities ranging from acute liver toxicity (Bradham et al., 1998; Kunstle et al., 1999) to shock syndrome with a lethal outcome (Natanson et al., 1989; Kircheis et al., 1992a, b). The reason for this is that in high doses TNF-α not only causes tumour necroses but is also the central mediator of endotoxic shock (Beutler et al., 1985; Hirota and Ogawa, 1999; Murphey et al., 2000). By contrast with locally limited tumour necrosis, in endotoxic shock, large amounts of TNF-α are released systemically. This leads to the failure of a range of regulatory functions of the body and results in life-threatening conditions such as hypotension, fever, metabolic acidoses, disseminated intravascular coagulopathies, as well as the loss of kidney, liver and lung function (Lenk et al., 1989; Kline et al., 1999; Mori et al., 1999; Ter 1998).

The pathogenesis proceeds via mechanisms which are entirely analogous to those which form the basis for the local inflammatory reaction or haemorrhagic tumour necrosis, except that these reactions are not locally limited here but occur systemically (Beutler and Cerami, 1986; Bendtzen, 1988; Kircheis, 1991); it includes widespread coagulopathies, diffuse capillary thrombosis, the escape of fairly large amounts of fluid, proteins and electrolytes from the blood into the tissues (Jahr et al., 1996; Hirota and Ogawa, 1999). These disturbances in the haemodynamics are potentiated by a reduced blood supply and disrupted metabolism and hence reduced performance of the heart (Natanson et al., 1989; Kline et al., 1999). The consequences are hypotension, the failure of vital organs, general disruption of the metabolism, further potentiated by catecholamines released in secondary manner. Small amounts of TNF-α also pass into the brain and by stimulating the synthesis of prostaglandin in the thermoregulating centre of the hypothalamus induce fever (Dinarello et al., 1986; Bendtzen, 1988).

Other tests on animals showed that doses of TNF-α with a marked antitumour activity already have significant toxicity and that even in the relatively TNF-α-resistant mouse there is only a small gap between the therapeutically active dose and the lethal dose, which means that in many cases systemic treatment with TNF-α would have no significant therapeutic benefit (Kircheis et al., 1992a; Kircheis et al., 1992b). In addition to the acute TNF-α-induced shock symptoms it was found that when TNF-α is permanently secreted into the bloodstream there is disruption to the fat metabolism (via the inhibition of key enzymes such as lipoprotein lipase), leading in extreme cases to wasting (cachexia) (Beutler et al., 1986).

In spite of acute and chronic TNF-α-induced toxicity, marked haemorrhagic tumour necroses with subsequent tumour regression were detected in some selected model systems after treatment with TNF-α.

The haemorrhagic tumour necroses observed in some cases led to various clinical trials into the use of TNF-α for treating malignant tumours. Studies on cancer patients however soon showed that the systemic use of TNF-α has serious side effects with little therapeutic benefit (Creaven et al., 1889; Lenk et al., 1989; Otto et al., 1990; Renard et al., 1994, 1995). In particular, acute toxic effects such as hypotension, disturbances in haemodynamics and liver toxicity were critical, while cachexia only proved to be a problem when given in long-term infusions, owing to the short half-life of TNF-α administered exogenously (Sherman et al., 1988). As a result of the limitation of the doses of TNF-α caused by its high toxicity and because of the short circulation times of TNF-α in the blood after systemic administration, the doses of TNF-α which finally reached the tumour were usually too small to induce any therapeutically effective antitumour effects.

These studies showed with great clarity that for effective clinical application it is necessary to reduce the toxicity of TNF-α and increase its antitumour activity (Haranaka, 1988; Kircheis, 1991). Attempts to separate the toxicity and antitumour activity by modifications at the TNF-α molecule have had little success (Kircheis et al., 1992a). The reasons for this are on the one hand the broad expression of the receptors for TNF-α on numerous normal tissues (Brockhaus et al., 1990), and on the other hand the fact that the antitumour effects, such as inflammatory reactions and haemorrhagic tumour necrosis, and the TNF-α-induced shock syndrome are based on the same pathophysiological mechanisms (Beutler and Cerami, 1986; Bendtzen, 1988; Kircheis, 1991; Anderson et al., 1994; Edwars et al., 1992; Ferrero et al., 1996; Renard et al., 1994, 1995; Westermann et al., 1999; Yi and Ulich, 1992). Although the sensitivity of the capillaries in the area of the tumour is greater than that of the capillaries in the normal tissue, because of the large number of normal tissues and vital organs damaged by TNF-α the desired therapeutic effect should be at least balanced out by undesirable toxic side effects.

Thus, for therapeutic use of TNF-α, it would appear to be necessary to separate the antitumour activity and systemic toxicity by striving for the maximum possible localisation or focussing of the effects of TNF-α on the tumour. By administering TNF-α locally directly into tumours it has been possible in some cases to induce visible antitumour effects without very much systemic toxicity (Jakubowski et al., 1989; Pfreundschuh et al., 1989; Van der Veen et al., 1999). However, the possibilities of direct administration into the tumour are severely limited in practice, particularly in the case of tumours or metastases in internal organs. In metastasising tumours such treatment could only be successful if all the metastases were amenable to direct administration. One particular method of administration is by the so-called “isolated limb perfusion” method in which the blood supply to whole limbs affected by tumours or metastases is uncoupled from the systemic blood supply for a certain time. This brief separate blood supply to the limbs makes it possible to administer higher doses of TNF-α sufficient to achieve a therapeutic effect and to restrict the toxic side effects to a relatively small part of the body (the limb in question) (Eggermont et al., 1996a, 1996b; Bickels et al., 1999; Vrouenraets et al., 1999). The advantage of this method of administration is mainly that vital TNF-α-sensitive organs such as the liver and lungs are largely spared the direct effects of TNF-α. In spite of the maximum possible protection of the internal organs from the direct effects of TNF-α even with this type of administration the doses of TNF-α cannot be set as high as desired because even in this case a certain amount of TNF-α will enter the systemic bloodstream (Stam et al. 2000). Moreover, the maximum dose which can be given is limited by the toxicity to normal tissue in the limbs, such as the capillary vascular system, muscles, etc. Attempts have been made to increase the therapeutic efficiency of this form of administration by combining TNF-α with IFNγ, as well as the cytostatic Melphalan (Eggermont et al., 1996a, 1996b; Lienard et al., 1999; Oliemann, 1999). The most serious disadvantage of this form of administration is its limited usefulness on restricted tumour locations (in the limbs).

However, in most cases of metastasising tumour diseases, the patients to be treated have tumour metastases even at difficult or inaccessible locations, or a plurality of widespread metastases which can only be reached through the bloodstream (often even only through the systemic bloodstream).

Apart from TNF-α, the related cytokines TNF-β, IL-1 and IL-6 have been considered for tumour therapy. TNF-β, or lymphotoxin, is closely related to TNF-α both in its evolution and functionally (Granger et al., 1968). TNF-α (157 amino acids) and TNF-β (171 amino acids) have 50% homology at the amino acid level) (Gray et al., 1984). Whereas TNF-α is secreted mainly by activated monocytes or macrophages, TNF-β is secreted mainly by NK cells, T-, B-lymphocytes. TNF-α and TNF-β bind to the same receptors (Hohmann et al., 1990) and have a virtually identical spectrum of activity (Gray et al., 1984; Kramer et al. 1986; Kircheis et al., 1992b). The spectrum of activity of TNF-α overlaps considerably with two other cytokines, namely with interleukin-1 and interleukin-6 (Bendtzen, 1988). As with TNF-α, IL-1 and IL-6 are inflammation mediators which also act on endothelial cells, macrophages, immune cells. IL-1, like TNF-α, induces fever in the hypothalamus and both cytokines may act as mediators in shock syndrome (Bentzen, 1988; Yi and Ulich, 1992; Mori et al., 1999; Hirota and Ogawa, 1999). The direct antitumour activity of IL-1 and IL-6, while having similar proinflammatory and immunostimulant activities, is weaker than that of TNF-α, and similarly the induction of haemorrhagic tumour necroses by these two cytokines is less typical. The problems relating to the systemic use of the TNF-α-related cytokines TNF-β, IL-1 and IL-6 occur because of the spectrum of activity which is similar or overlapping with TNF-α.

The direct administration of TNF-α or the TNF-α-related cytokines TNF-β, IL-1 and IL-6 is generally only possible if the disease is restricted to a primary tumour and if this tumour is accessible for direct administration. In most cases, however, the diseases being treated are metastasising tumours which are for the most part located in the visceral organs and are therefore not directly accessible. In these cases the tumours are amenable to treatment with the effective protein only via a systemic administration route. However, as already explained, in the case of the proteins this administration is connected with systemic toxicity. Moreover, owing to the short half-lives of the therapeutically effective proteins in the blood the concentrations which are finally available at the tumour itself are too low to produce the desired therapeutic effects.

Alternatively, it was therefore proposed to administer, instead of the proteins, the DNA molecules coding therefor, the crucial point being that the expression of the protein, excluding normal tissues at risk from toxicity, should take place exclusively at the target site, namely at the tumour, if possible. Various approaches for targeted gene expression in tumours have already been proposed, e.g. targeting of the TNF-α gene by means of a conjugate of a synthetic TNF-α gene and an antibody against a cell surface protein repeatedly expressed on tumour cells, e.g. an anti-transferrin receptor antibody (Hoogenboom et al., 1991), or by means of a TNF-α gene encapsulated in fusogenic liposomes (Mizuguchi et al., 1998). Alternatively, it was proposed to bring about targeted expression of TNF-α by means of a tissue- and cell cycle-specific promoter (Jerome and Muller, 1998).

In therapy using DNA, under normal conditions, the administration of unprotected DNA, e.g. in the form of a plasmid, into the bloodstream leads to rapid inactivation and breakdown of the DNA. One approach used for protecting the DNA from being broken down too fast consists in complexing the DNA with positively charged polycations (Boussif et al., 1995; Abdallah et al., 1996; Goula et al., 1998), or, alternatively, cationic lipids (Song et al., 1997; Liu et al., 1997; Li and Huang, 1997; Templeton et al., 1997; Liu et al., 1997; Lee et al., 1996). Condensation of the DNA by the polycation forms compact particulate DNA complexes which largely protect the DNA from breakdown and make it easier to absorb into the cells. Effective, i.e. most complete condensation of the DNA generally takes place, however, when there is an excess of polycation compared with the DNA, i.e. when there is an excess of positive charge (Boussif et al., 1995; Liu F, 1997; Liu Y, 1997). The resulting DNA complex is therefore also positively charged. Apart from its significance to the condensation of the DNA the positive charging also makes it possible for the DNA complex to bind to negatively charged structures on the cell surface (by electrostatic adsorption) and for the DNA complex to be taken up subsequently by adsorptive endocytosis. The positive charging of the polycation/DNA complexes does however present a serious problem for systemic administration in vivo, as it also leads to a broad palette of non-specific electrostatic interactions with blood components, extracellular matrix and non-target cells (Plank et al., 1996; Ogris et al., 1999; Kircheis and Wagner, 2000). In the event of administration in vivo this irrevocably leads to recognition by the complement system (Plank et al., 1996) and by the reticuloendothelial system (Gregoriadis, 1988; Mahato et al., 1995), followed by rapid inactivation and breakdown. Moreover, the non-specific uptake into non-target calls leads to unwanted, uncontrolled gene expression, combined with uncontrolled biological effects and toxicity (Kircheis et al., 1999).

The systemic administration of polycation/DNA complexes wherein the positive charge is not screened leads to non-specific gene expression in various vital organs, mainly the lungs and liver, and to toxicity, resulting in acute pulmonary embolism and death in the most serious cases (Kircheis et al., 1999), as was shown by means of the luciferase reporter gene. The assays carried out in vitro showed non-specific interactions of positively charged polycation/DNA complexes with plasma proteins such as fibrinogen and the complement system, and the aggregation of erythrocytes would appear to be responsible inter alia for these toxic effects in vivo (Ogris et al., 1999; Kircheis et al., 1999; Kircheis and Wagner, 2000). If the luciferase reporter gene, which is categorised as biologically inert, is replaced by a TNF-α-coding gene, potentiation of the toxicities of gene transfer-induced toxicity and TNF-α-mediated toxicity must be expected, while in addition the high gene expression in the lungs and liver would prove particularly unfavourable, as these organs are particularly sensitive to TNF-α-mediated toxicity (Lenk et al., 1989; Bradham et al., 1998; Kunstle et al., 1999; Mori et al., 1999).

Attempts have previously been made to screen the positive surface charge of polycation/DNA complexes, which can be determined by physical measurement of the zeta potential (Müller R H, 1996), and the resulting non-specific interactions, by means of a polyethyleneglycol shell (Ogris et al., 1999; Kircheis et al., 1999; WO 98/59064).

Moreover, Dash et al., 2000, describe a method of screening polylysine/DNA complexes by means of a polymethacrylic polymer (pHPMA).

The aim of the present invention was to overcome the problems which arise in the systemic use of DNA in the therapy of tumour diseases, particularly the problem of non-specific expression in normal tissue and the toxicity which may be connected thereto, e.g. in the case of TNF-α, by preparing a new system of administration for DNA.

In solving this problem the primary consideration, with regard to the therapeutic effect of therapeutically effective proteins with a cytotoxic activity, particularly TNF-α and/or the TNF-α-related cytokines TNF-β, IL-1 and IL-6, after the administration of the DNA coding for these cytokines into the bloodstream or through the systemic circulation of the blood, was to bring about gene expression and the resulting cytokine-mediated effects in targeted manner, i.e. specifically targeted to the tumour tissue, while sparing the normal tissue as far as possible. This targeted direction to the tumour tissue is hereinafter referred to as “Tumour Targeting”.

The present invention relates to a complex for the treatment of tumour diseases by systemic administration of DNA, containing, in expressible form, one or more DNA molecules, coding for one or more therapeutically active proteins with a cytotoxic activity, and a polycation which condenses the DNA and is wholly or partly conjugated with transferrin, characterised in that the complex has a surface charge which corresponds to a zeta potential of ≦+15 mV, obtained by measuring in aqueous solution at a concentration of ≧10 mM NaCl, more than 50% of the screening of the positive charges in the complex being effected by transferrin.

Preferably, the complexes have a zeta potential of +10 mV to −10 mV, most preferably +5 mV to −5 mV.

“Systemic administration” for the purposes of the present invention includes not only systemic administration through the entire circulation of the blood but also regional application through the blood vessels supplying the tumour, i.e. any form of administration which is not directly into the tumour but via the bloodstream.

By “a cytotoxic activity” is meant a direct cytotoxic activity of the protein (e.g. as in the case of TNF-α), but also an indirect activity, as obtained for example by the release of a cytotoxic substance from a non-toxic substrate brought about by the enzymatically active protein, corresponding to the activity of the so-called suicide gene.

The zeta potential can be determined by standard methods, e.g. as described by Müller R H, 1996.

Preferably, the DNA codes for TNF-α and/or for TNF-β and/or IL-1 and/or IL-6, of which TNF-α is particularly preferred.

Also suitable within the scope of the present invention are DNA molecules coding for other proteins with an antitumour activity and a cytotoxic activity, e.g. selected from among the cytokines IFN-α, IFN-γ, or toxins such as the diphtheria toxin (Massuda et al., 1997); also so-called suicide genes (Aghi et al., 2000), which are used in conjunction with the substrate, such as the Herpes Simplex thymidine kinase gene (with ganciclovir; Nagamachi et al., 1999), cytochrome P450 (with cyclophosphamide) (Aghi et al., 2000), or the linamarase gene (with linamarin; Cortes et al., 1998).

The DNA molecules coding for cytotoxic anti-tumour proteins may be used individually or in combination, e.g. a TNF-α-plasmid in conjunction with a plasmid, coding for a suicide gene, e.g. the thymidine kinase gene.

The DNA coding for a protein with a cytotoxic activity may be combined with one or more other DNA molecules which code for a protein with an anti-tumour activity, e.g. for an immunotherapeutically active cytokine such as interleukin-2 or interferon-gamma, for an apoptosis-inducing protein such as p53 (Xu et al., 1999) or apoptin, for a caspase, for FasL (FasLigand) (Gajate et al., 2000) or for inhibitors of neoangiogenesis in the tumour such as endostatin (O'Reilly et al., 1997); angiostatin (Griscelli et al., 1998), or Kringle 1-5 (Cao et al., 1999).

The expression plasmid must satisfy the requirement of being suitable for expression in mammalian cells. Preferably, it contains a strong promoter, e.g. the CMV promoter or the SV-40 promoter, which guarantees the strong expression which is required for the therapeutic activity. In another preferred embodiment, expression plasmids may be used which ensure tumour-specific expression, e.g. using a tumour-specific, cell cycle-specific or tissue-specific promoter, or by hypoxia-responsive elements; in addition, regulatory elements which can be induced physically (by radiation) or chemically (e.g. by tetracycline) are also suitable (Jerome, et al., 1993;, Dachs et al., 1997).

When a number of proteins are used the complex preferably contains a plurality of expression plasmids each coding for a single therapeutic protein.

In one embodiment of the invention the DNA sequence coding for the therapeutic protein is preceded by a leader sequence which allows the protein to be secreted. Examples of suitable leader sequences are Type I- and Type II leader sequences, e.g. in the case of TNF-α the endogenous TNF-α Type II leader sequence (Utsumi et al., 1995). Type II leader sequences typically consist of a cytoplasmic part, a transmembrane domain and a linker domain which is adjacent to the mature protein. In the case of TNF-α the endogenous pro-TNF-α leader sequence is 76 amino acids long; using it means that the transfected cells can correctly process the pro-TNF-α form.

Alternatively to a Type II leader sequence the coding sequence may be preceded by another leader sequence which brings about the secretion of the protein, e.g. a human Type I immunoglobulin leader sequence. The Type I leader sequences, which have been described for numerous proteins (von Heijne, 1983), are secretory leaders not more than 18-23 amino acids long. These Type I leader sequences cause the proteins to bind to the endoplasmic reticulum, where the proteins are subsequently transported through the membrane, cleaving the leader. Examples of suitable leader sequences within the scope of the present invention are e.g. immunoglobulin leader sequences, such as Acc.No. AF174024.1, which are described in the Kabat data bank, or synthetic immunoglobulin leaders consisting of a consensus leader sequence derived from the immunoglobulin leader sequences described above.

The use of an endogenous Type II leader sequence has the advantage that the secretion can take place to a lesser extent, optionally with some delay, compared with Type I leader sequences, which is particularly advantageous in the case of a toxic protein. In cases where a high toxic concentration is required, Type I leader sequences may be superior to the Type II leader sequences.

Within the scope of the present invention, all polycations which perform the function in the complex of balancing out the negative charge of the plasmid DNA and condensing the plasmid DNA into compact particles are suitable.

Examples of polycations are polyethyleneimines (PEI), homologous polycationic polypeptides such as polylysine, polyarginine, histones, spermines, spermidines, cationic lipids, dendrimers (Boussif et al., 1995; Abdallah et al., 1996; Goula et al., 1998 a, b; Haensler, et al., 1993; Kukowska-Latallo, et al., et al. 1996; Lee, et al., 1996; Li, et al. 1997; Liu, et al., 1997; Felgner, et al., 1994; Fritz, et al., 1996; Schwartz, et al., 1995; Tang, et al., 1996; Thurston, et al., 1998; Van de Wetering, et al., 1998; Wagner, et al., 1990; Wagner, et al., 1991).

Preferably, the complex according to the invention contains a polyethyleneimine (PEI)as polycation.

The PEI may have a linear or branched structure, and the molecular weight range may vary over a wide range, namely between about 0.7 kDa and about 2000 kDa, preferably about 2 kDa to about 50 kDa.

Larger PEI molecules often lead to greater condensation of the DNA and after complexing with DNA yield an optimum transfection efficiency even at low N/P ratios, they generally result in very good transfection efficiency but may also be associated with a degree of toxicity. Smaller molecules, which are required in a larger amount for the amount of DNA specified, have the advantage of lower toxicity, albeit with possibly lower efficiency. The particular PEI molecule to be used in any one case can be determined by preliminary tests.

Particularly preferred within the scope of the present invention are PEI molecules with an average molecular weight range of between 2000 D and 800,000 D.

Examples of commercially obtainable PEI with different molecular weights which is suitable within the scope of the present invention include PEI 700 D, PEI 2000 D, PEI 25000 D, PEI 750000 D (Aldrich), PEI 50000 D (Sigma), PEI 800000 D (Fluka). BASF also market PEI under the brand name Lupasol® in different molecular weights (Lupasol® FG: 800 D; Lupasol® G 20 anhydrous: 1300 D; Lupasol® WF: 25,000 D; Lupasol® G 20: 1300 D; Lupasol® G 35: 2000 D; Lupasol® P: 750,000 D; Lupasol® PS: 750000 D; Lupasol® SK: 2,000,000 D).

Transferrin coupled to the polycation, i.e. human transferrin, which acts both as a ligand for cell binding and to screen non-specific interactions with non-target cells or non-target structures, is preferred (Wagner et al., 1993; Kircheis et al., 1997).

The transferrin may be coupled to the polycation in the conventional way, e.g. as described in EP 388 758 or WO 92/19281 for the preparation of transferrin-polycation conjugates.

The following procedure is expediently used to determine the composition of the complex: Starting from a defined amount of DNA which is present, for example, in the form of a reporter gene plasmid (luciferase, beta-gal-plasmid), the amount of polycation added is titrated in test series, specifically with a view to optimum condensation of the DNA into compact particles, maximum transfection efficiency into tumour cells and minimum cytotoxicity.

If PEI is used as the polycation the ratio of DNA to PEI hereinafter is given by the molar ratio of the nitrogen atoms in the PEI to the phosphate atoms in the DNA (N/P value or N/P ratio); an N/P value of 6.0 corresponds to a mixture of 10 μg DNA with 7.5 μg of PEI. In the case of free PEI only about every sixth nitrogen atom is protonated under physiological conditions. Results with DNA/PEI transferrin complexes show that these are approximately electroneutral at an N/P ratio of 2 to 3.

The N/P value of the complexes may vary over a wide range; it may be in the range from about 0.5 to about 100. Preferably, the N/P ratio is about 2 to about 20, and most preferably the ratio is 4 to 10.

Specifically the N/P value for the particular application, e.g. for the type of cell to be transfected, can be determined by preliminary tests, by increasing the ratio, under otherwise identical conditions, in order to determine the optimum ratio with respect to transfection efficiency and to rule out any toxic effect on the cells.

Within the scope of the present invention the efficiency of complexes which contain linear PEI with a molecular weight of 22 kDa and branched PEI with a molecular weight of 25 kDa is shown.

The formulation of the complex according to the invention is further selected so that the positive charge of the polycation-transferrin/DNA complex is largely screened from the high transferrin component in the polycation conjugate, corresponding to the zeta potential of ≦+15 mV.

The ratio of quantities of transferrin/polycation is determined to suit the particular polycation by carrying out series of tests with a given ratio of DNA/polycation in order to titrate the amount of conjugated transferrin-polycation conjugate with a different polycation/transferrin ratio in series of tests, with a view to obtaining optimum condensation of the DNA into compact particles, maximum transfection efficiency into tumour cells and minimum cytotoxicity. An essential parameter for the choice of the polycation/transferrin ratio is the screening of the surface charge of the complex by the transferrin contained in the conjugate, corresponding to a zeta potential of ≦+15 mV.

The ratio of transferrin:polycation in the transfection complex finally obtained is preferably 3:1 to 1:4 (w/w).

The proportion of non-transferrin-conjugated polycation (“free polycation”) in the complex is dependent on the molecular weight of the polycation and may be in the range between 0% and 95% (molar ratio) of the total polycation content.

The smaller the polycation molecule, the more appropriate it is to dilute with free polycation. For example, when using PEI 800 kDa, e.g. when using the conjugate Tf8PEI800 kDa (conjugate with a molar ratio of 8 transferrin molecules per PEI molecule, branched, with an average molecular weight of 800 kDa, see Kircheis et al., 1997) no additional free PEI is needed, whereas when Tf-PEI25 kDa is used (conjugate with a molar ratio of transferrin:PEI=1, branched, with an average molecular weight of 25 kDa, cf. the present invention) dilution with 2-10-times the amount of PEI25 or PEI22 is advisable (particularly preferably dilution with 3-5-times the amount of PEI25 or PEI22).

The molar ratio of conjugated polycation:free polycation is preferably about 1:0 to 1:20.

The polycation conjugated with transferrin may be identical to any free polycation present in the complex, but the polycations may also be different.

Further requirements of the complex are that it should be well tolerated when administered into the systemic circulation of the blood (even in experimental animals, e.g. tumour-bearing mice), and that it should give maximum gene expression in the tumour with the least possible expression in vital normal tissues such as the liver, lungs, kidneys, spleen and heart.

In a preferred embodiment the positive charge is totally screened by the high transferrin content of the conjugate. If transferrin is only predominantly responsible for the screening, the following should be borne in mind: although the majority of the screening (>50%) of the positive charge of the complex is done by the high transferrin content of the conjugate, the reduction in the zeta potential may, to a lesser extent, be achieved by specifically adapting other complex parameters, e.g. by reducing the N/P ratio or incorporating negatively charged molecules into the formulation, e.g. negatively charged fusogenic peptides as described for example in WO 93/07283 (peptides of this kind simultaneously have the effect of causing the complexes to be released from the endosomes, cf. the next paragraph).

Other substances which may be present in the complex to help to screen the positive charge, in addition to transferrin, are hydrophilic polymers, e.g. polyethyleneglycols (PEG), polyvinylpyrollidones, polyacrylamides, polyvinylalcohols, or copolymers of these polymers. PEG is the preferred hydrophilic polymer. The molecular weight of the hydrophilic polymer is generally about 1,000 to about 40,000 Da; molecules with a molecular weight of 5,000 to 40,000 Da are preferably used.

Preferably, the hydrophilic polymer, preferably PEG, is covalently bound to the polycation, particularly PEI. The covalent binding is effected either by conjugation with the free polycation, particularly PEI, or by incorporation into the transferrin-PEI conjugate. In the latter case the hydrophilic polymer is arranged between transferrin and the polycation; a molecule of this kind is obtained by using a bifunctional polymer which has different reactive groups at both ends of the molecule and reacting it with transferrin on the one hand and with the polycation on the other hand. Examples of bifunctional polymers of this kind include e.g. PEGs, as used hitherto for crosslinking various macromolecules, e.g. for crosslinking cofactor and apoenzyme (Nakamura et al, 1986), the targeting of polymeric active substances (Zalipsky and Barany, 1990) or PEG coating of surfaces and proteins (Harris et al, 1989). The bifunctional derivatives which may be used inter alia within the scope of the present invention are commercially obtainable; they contain amino groups, hydroxy groups or carboxylic acid groups at the ends of the molecule, e.g. like the products obtainable from Shearwater Polymers.

The contribution which these hydrophilic polymers make to the screening of the positive charge is less than 50%, preferably not more than 30%.

The proportion of screening by transferrin compared with the screening effects achieved by other factors can be determined by measuring the zeta potential of complexes with a plurality of screening factors with or without a transferrin content, the contribution of the transferrin being obtained from the difference in the zeta potentials.

In the interests of optimum gene expression the complex may also contain elements to increase the release of the DNA complexes from the endosomes of the target cell, e.g. fusogenic peptides (WO 93/07283), or elements which intensify the uptake of DNA into the cell nucleus, such as so-called nuclear targeting sequences (Vacik, et al., 1999; Zanta, et al., 1999).

In another aspect the present invention relates to a pharmaceutical composition containing one or more of the complexes according to the invention. In this composition the complexes are preferably taken up in an isotonic aqueous solution, e.g. in 0.5×HBS (HEPES (20 mM) -buffered saline solution (75 mM NaCl) with 2.5% glucose, as described in the examples. In other preferred applications, the complexes are taken up in aqueous solutions in a wide range of salt concentrations (0-150 mM NaCl), concentrations of the HEPES-buffer (0-1M) and also with other buffer systems (phosphate buffer, etc.). The isotonicity of the solutions can generally be obtained by either a suitable salt content (1.50 mM NaCl=isotonic) or by a corresponding sugar content (5% glucose or 10% sucrose-isotonic), or by the addition of corresponding amounts of salt and sugar (e.g. 75 mM NaCl and 2.5% glucose).

In the course of the experiments carried out (cf. Example 10) it was discovered that at a low concentration of the complex in the formulation or with a smaller amount of the formulation finally administered (cf. Example 10 and Example 9 or 8) the smaller amount of DNA administered can be compensated in the formulation by an increased salt concentration (≦80 mM, preferably ≧100 mM, particularly ≧150 mM NaCl, the maximum salt concentration being about 1M). It has been found that the efficiency of a formulation in which the amount of DNA has been reduced to 10%, when the salt concentration was doubled from 75 mM to 150 mM, was approximately the same as that of a formulation with 10 times as much DNA which has been mixed at a lower salt concentration, and both formulations resulted in a significant inhibition of tumour growth (cf. Example 10 compared with Examples 8 and 9).

In another embodiment the formulations of the complexes according to the invention were stored deep frozen in aqueous solution and thawed before use, or stored in lyophilised form, which enables them to be stored under stable conditions for lengthy periods, and reconstituted before use in one of the saline buffer solutions described.

By contrast with gene transfer systems in which the positive charge of the polycation is not screened (Zeta potential >+20 mV) (Goula et al., 1998b; Song et al., 1997; Liu et al., 1997; Li and Huang, 1997; Templeton et al., 1997; Liu et al., 1997; Lee et al., 1996), the complexes according to the invention are capable of specifically bringing about the expression of a therapeutically active gene (e.g. coding for TNF-α) after administration into the bloodstream.

Within the scope of the invention it has been shown that the use of an expression plasmid coding TNF-α in a tumour-targeted gene transfer system in syngenic tumour models in the mouse brings about the haemorrhagic tumour necrosis typical of TNF-α, followed by a significant inhibition of tumour growth, but without any systemic TNF-α-mediated toxicity of the kind known from systemic administration of TNF-α.

It has been found that the incorporation of a large enough amount of transferrin in the polycation/DNA complex can also screen the positive surface charge. By screening the positive surface charge, the non-specific electrostatic interactions are also screened. The incorporation of a large enough quantity of transferrin inhibits the aggregation of erythrocytes, whereas unscreened polycation/DNA complex leads to a major aggregation of erythrocytes.

Systemic administration of screened gene transfer complexes of this kind (the phrase “screened complexes” is hereinafter used for simplicity's sake to refer to complexes in which the positive charge is screened, predominately by transferrin) through the caudal vein in the mouse leads to predominant gene expression in the tumour (illustrated by the example of the luciferase reporter gene) and negligible expression in other organs. Unlike unscreened gene transfer complexes, no systemic toxicity was observed.

Tests with these gene transfer complexes which are capable of specifically expressing a reporter gene in the tumour constituted the basis for further tests in which expression plasmids which code for the biologically highly active TNF-α were administered into the bloodstream by systemic route.

After repeated administration, haemorrhagic tumour necrosis was specifically found in the tumour in 60-70% of the animals treated (experiments corresponding to FIGS. 5a and 6a). Haemorrhagic tumour necrosis, one of the characteristics of the anti-tumour TNF-α activity, resulted in the killing off of large parts of the affected tumours and subsequent significant inhibition of tumour growth (experiments according to FIGS. 5b and 6b). The TNF-α-mediated effects observed were focused specifically on the tumour without any apparent systemic toxicity of the kind known to result from administration of systemic TNF-α (protein) (Beutler and Cerami, 1986; Bendtzen, 1988; Haranaka, 1998; Natanson et al., 1989; Lenk et al., 1989; Kircheis et al., 1992) (Experiments corresponding to FIGS. 5 and 6). Tumour necrosis after the administration of the TNF-α gene transfer systems was found in tumours of completely different histological origins (e.g. Neuro2a neuroblastoma, MethA fibrosarcoma).

By using the tumour-targeted system for the gene transfer of the TNF-α gene it is possible to achieve TNF-α-mediated anti-tumour activities such as haemorrhagic tumour necrosis and inhibition of tumour growth even after systemic administration into the bloodstream, without the features of a systemic TNF-α-mediated toxicity of the kind known from the systemic administration of TNF-α.

The complexes according to the invention or the pharmaceutical compositions containing them may be used to treat diseases which are associated with solid tumours, particularly metastasising tumours of malignant melanoma, soft tissue sarcoma, fibrosarcoma, adenocarcinomas of the gastrointestinal tract, colon carcinoma, liver cell carcinoma, pancreatic cancer, lung cancer, breast cancer, osteosarcomas, glioblastoma and neuroblastoma.

The use of the tumour-targeted gene transfer complexes according to the invention, containing a therapeutically active gene with a cytotoxic effect or a combination of such a gene with one or more genes by systemic gene therapy may advantageously be combined with conventional standard therapies for treating cancer such as chemotherapy (Blumenthal et al., 1994) or radiotherapy (Xu et al., 1999; Rosenthal et al., 1999).

Examples of chemotherapeutic agents which may be used in conjunction with the complexes according to the invention (by administration beforehand, simultaneously or afterwards) are doxorubicin, taxol, 5-fluorouracil, cisplatin, vinblastin or formulations of these chemotherapeutic agents, e.g. doxil (liposomal formulation of doxorubicin). Preferably, the chemotherapeutic agent is administered in a dose which is lower and therefore better tolerated than the dose conventionally used when this therapeutical agent is used in monotherapy. Within the scope of the present invention it has been shown that combining a complex according to the invention which contains TNF-α-DNA with doxil was more effective then monotherapy with doxil. This effect was particularly marked if doxil was administered in a lower dose than the maximum dose which could be administered, restricted on account of its toxicity (cf. Example 15 with Example 14).

### SUMMARY OF THE FIGURES

FIG. 1: Non-specific gene expression in various organs and systemic toxicity in the systemic administration of positively charged polycation/DNA complexes into the bloodstream

FIG. 2: Aggregation of erythrocytes by positive surface charge of polycation/DNA complexes

FIG. 3: Screening of positive surface charge and associated inhibition of erythrocyte aggregation by incorporation of a high transferrin content into polycation/DNA complexes

FIG. 4: Systemic administration of screened transferrin-containing polycation/DNA complexes

FIG. 5: Systemic administration of screened transferrin-containing polycation/DNA complexes in a neuroblastoma model

FIG. 6: Systemic administration of screened transferrin-containing polycation/DNA complexes in a fibrosarcoma model

FIGS. 7-10: Systemic administration of screened transferrin-containing polycation/DNA complexes in a neuroblastoma model

FIG. 11: Systemic administration of screened transferrin-containing polycation/DNA complexes in the melanoma model M-3

FIG. 12: Systemic administration of screened transferrin-containing polycation/DNA complexes in the melanoma model B16F10

FIGS. 13 and 14: Systemic administration of screened transferrin-containing polycation/DNA complexes containing TNF-α plasmid-DNA, in conjunction with a chemotherapeutic agent

FIG. 15: Screened transferrin-containing polycation/DNA complexes can be safely stored for lengthy periods.

The following materials and methods were used in the Examples that follow, unless otherwise indicated:

Materials

1) Starting Materials for the Transferrin-Polycation Conjugates

a) PEI

Polyethyleneimine (PEI) with a molecular weight of 25 kDa, (PEI(25)) was obtained from Aldrich, Milwaukee, Wis.

PEI with a molecular weight of 800 kDa, PEI(800) (in the form of a 50% w/v solution) was obtained from Fluka, Buchs.

Linear PEI with a molecular weight of 22 kDa (PEI(22)) was obtained from Euromedex, Soufferlweyersheim, France, or from MBI Fermentas, St. Leon-Rot, Germany.

b) Human transferrin (iron-free) was obtained from Biotest, Dreieich, Germany.

2) Preparation of a Transferrin-PEI(25) Conjugate

A transferrin-PEI(25) conjugate was synthesised as described in Kircheis et al., 1997, with some modifications:

A solution of PEI(25 kDa) in the form of the HCl salt in water was gel-filtered through a Sephadex G-25 Superfine chromatography column (Pharmacia, Uppsala).

Liquid chromatography was carried out using a Merck-Hitachi L-6220 pump and a L-4500A UV-VIS detector. The amount of PEI in the individual fractions was determined using the ninhydrin test and measured by spectrophotometry at 570 nm. The quantity of transferrin was determined by spectrophotometry at 280 nm.

A solution of transferrin in 150 mM NaCl was purified by gel filtration through a Sephadex G-25 Superfine chromatography column (Pharmacia, Uppsala) against 30 mM sodium acetate buffer (pH 5). The gel-filtered transferrin was cooled to 0° C., and 3 equivalents of sodium periodate in 30 mM sodium acetate buffer (pH 5) were added. The reaction mixture was incubated on ice for 90 minutes in the dark. Low-molecular by-products were separated off by gel filtration through a Sephadex G-25 Superfine chromatography column (with 30 mM sodium acetate buffer, pH 5). The oxidised transferrin [quantity determined by UV absorption at 280 nm] (in 30 mM sodium acetate buffer, pH 5) was immediately (within 15 minutes) added to the PEI(25) solution (in 0.25 M sodium chloride) in a molar ratio of 1:1.2 with vigorous mixing and incubated for 30 minutes at ambient temperature. The pH of the reaction mixture was adjusted to 7.3 by the addition of 2M HEPES pH 7.9. Then 4 batches of sodium cyanoborohydride (1 mg per 10 mg of transferrin) were added at intervals of 1 hour. After 19 hours the salt concentration was adjusted to 0.5M salt by the addition of 3M sodium chloride. The reaction mixture was added to a cation exchange chromatography column (Bio-Rad Macro-Prep high S) and fractionated with a gradient of 0.5-3.0M sodium chloride (with a constant content of 20 mM HEPES pH 7.3). The coupling product was eluted at a salt concentration of between 2.0M and 3.0M. After dialysis with 21 HBS pH 7.3 (150 mM sodium chloride, 20 mM HEPES pH 7.3) the conjugate (known as: Tf-PEI) was obtained with a molar ratio of transferrin:PEI=1/1.0-1.1. The concentration was adjusted to 1 mg PEI/ml by the addition of HBS, pH 7.3.

The iron was incorporated by the addition of 1.25 μL of 10 mM iron (III) citrate buffer (containing 200 mM citrate, adjusting the pH to 7.8 by the addition of sodium bicarbonate) per 1 mg of transferrin content. The Tf-PEI conjugate in its ferruginous form was divided into suitable aliquots which were deep-frozen in liquid nitrogen and stored at −80° C.

3) Plasmids

a) The luciferase reporter gene plasmid used was the plasmid pCMVL (also known as pCMVLuc) described in WO 93/07283.

b) TNF-α Plasmids

i) Expression Plasmids Coding for Murine TNF-α with an Endogenous Leader Sequence

The DNA insert for murine TNF-α with the endogenous leader was prepared by overlapping PCR reactions. The leader sequence was amplified by exon 1 and exon 2 of genomic mouse DNA. The sequence coding for mature TNF-α was amplified by TNF-α cDNA. The cDNA was obtained from RNA from LPS-activated monocytes by RT-PCR. The individual fragments were joined together by an overlapping PCR reaction (splice overlap reaction).

Specific cloning sites were inserted by PCR primers. The DNA sequence of the insert was determined and compared with the TNF-α sequence in Acc. No. NM013693.

The following PCR reactions were carried out:

1) Amplification of exon 1 with the PCR primers SEQ ID NO:1 and SEQ ID NO:2 with genomic mouse DNA as the template. SEQ ID NO:1 contains XhoI and BglII cloning sites.

2) Amplification of exon 2 with the PCR primers SEQ ID NO:3 and SEQ ID NO:4 with genomic mouse DNA as template.

3) Amplification of the mature TNF-α with the PCR primers SEQ ID NO:5 and SEQ ID NO:6 ; and mouse cDNA as template. The primer SEQ ID NO:6 contains the cloning sites KpnI and BamHI.

All 3 PCR fragments were joined to the end primers SEQ ID NO:1 and SEQ ID NO:6 in a combined PCR reaction (splice overlap reaction). The resulting fragment was cloned into the eukaryotic expression plasmids pGS-hIL-2 (tet) (Buschle et al., 1995) and pWS2m (Schmidt et al., 1995).

The pGS-hIL-2 (tet) vector was digested with BglII and KpnII and the hIL-2 insert was exchanged for the murine TNF-α insert, which was also digested with BglII-KpnI. The resulting plasmid was named pGS-muTNF-α.

The pWS2m vector was digested with BamHI and the muIL-2 insert was exchanged for the murine TNF-α insert, which was digested with BglII-BamHI. After checking the correct orientation of the insert the resulting plasmid was named pWS2-muTNF-α.

ii) Expression Plasmids, Coding for Murine TNF-α with Immunoglobulin Leader

Synthetic oligonucleotides for human immunoglobulin leader sequences were prepared according to the sequence Acc. No. AF174024.1 or according to a synthetic leader sequence and in a PCR reaction placed before the mature TNF-α sequence. The DNA sequence for the synthetic immunoglobulin leader corresponds to the sequence of Acc. No. Z69026.1 in the first 53 nucleotides plus acgatgt before the sequence of the mature TNF-α. In a PCR reaction the leader sequence in question was fused to the mature TNF-α.

In order to fuse the immunoglobulin leader according to the sequence of Acc. No. AF174024.1 to murine TNF-α, the following PCR reaction was carried out:

Amplification of the mature murine TNF-α with the PCR primers SEQ ID NO:7 and SEQ ID NO:6, using TNF-α cDNA as the DNA template. SEQ ID NO:7 contains XhoI and BglII restriction sites. The resulting fragment was cloned into the plasmids pGS-hIL-2 (tet) and pWS2m.

The pGS-hIL-2 (tet) vector was digested with BglII and KpnII and the hIL-2 insert was exchanged for the murine TNF-α insert with IgG leader, which was also digested with BglII-KpnI. The resulting plasmid was named pGS-muTNF-αIgL.

The pWS2m vector was digested with BamHI and the muIL-2 insert was replaced by the murine TNF-α insert with IgG leader, which had previously been digested with BglII-BamHI. After checking the correct orientation of the insert the resulting plasmid was named pWS2-muTNF-αIgL.

To fuse the synthetic immunoglobulin leader to murine TNF-α, the following PCR reaction was carried out:

Amplification of the mature murine TNF-α with the PCR primers SEQ ID NO:8 and SEQ ID NO:6, the DNA template used was TNF-α cDNA. SEQ ID NO:8 contains XhoI and BglII restriction sites. The resulting fragment was cloned into the plasmids pGS-hIL-2 (tet) and pWS2m.

The pGS-hIL-2 (tet) vector was digested with BglII and KpnII and the hIL-2 insert was exchanged for the murine TNF-α insert with synthetic IgG leader, which was also digested with BglII-KpnI. The resulting plasmid was named pGS-muTNF-α-sIgL.

The pWS2m vector was digested with BamHI and the muIL-2 insert was exchanged for the murine TNF-α insert with synthetic IgG leader, which had previously been digested with BGlII-BamHI. After checking the correct orientation of the insert the resulting plasmid was named pWS2-muTNF-α-sIgL.

### Example 1

Non-Specific Gene Expression in Various Organs and Systemic Toxicity in the Systemic Administration of Positively Charged Polycation/DNA Complexes into the Bloodstream

Transfection complexes, consisting of PEI and DNA (in the form of an expression plasmid coding for the luciferase reporter gene) were prepared in 75 mM NaCl, 20 mM HEPES at a DNA concentration of 200 μg/ml.

PEI(800)/DNA complexes (N/P=6, N=nitrogen of the polycation, P=phosphate of the DNA) were prepared by rapidly mixing a PEI(800) solution (156 μg PEI(800)/ml in 75 mM NaCl, 20 mM HEPES) and a solution of the DNA in a concentration of 200 μg DNA/ml in 75 mM NaCl, 20 mM HEPES.

PEI(25)/DNA complexes (N/P=4.8) were prepared by rapidly mixing a PEI(25) solution (125 μg PEI(25)/ml in 75 mM NaCl, 20 mM HEPES) and a solution of the DNA (200 μg DNA/ml in 75 mM NaCl, 20 mM HEPES).

PEI(22)/DNA complexes (N/P=4.8) were prepared by rapidly mixing a PEI(22) solution (125 μg PEI(22)/ml in 75 mM NaCl, 20 mM HEPES) and a solution of the DNA (200 μg DNA/ml in 75 mM NaCl, 20 mM HEPES).

The transfection complexes were incubated for 20 minutes at ambient temperature after mixing. To ensure isotonicity, glucose was added (to give a final concentration of 2.5%).

The particle size of the PEI/DNA complexes was measured in the standard way using a Malvern Zetasizer 3000.

The surface charge of the PEI/DNA complexes (1:30 diluted in a 10 mM NaCl solution) was determined by physical measurement of the zeta potential using a Malvern Zetasizer 3000. (The method of measuring the zeta potentials is described in Müller R H, 1996, Zetapotenzial und Partikelladung in der Laborpraxis, Wissenschaftliche Verlagsgesellschaft W V G Stuttgart.)

The transfection complexes (containing 50 μg DNA/250 μl) were administered systemically through the caudal vein into tumour-bearing syngenic A/J mice (neuroblastoma, Neuro2a, growing subcutaneously in the flank) (with at least 4 animals per test group). For this purpose the mice had been injected subcutaneously 2 weeks previously with 10⁶ Neuro2a tumour cells (ATCC CCL 131), and at the time of the administration of the transfection complexes had subcutaneously growing tumours with diameters of 10-13 mm.

A control group of mice was injected with an equal amount of non-condensed DNA (50 μg DNA/250 μl).

The reporter gene expression was measured 24 hours after administration of the transfection complexes by means of a luciferase assay (described in Kircheis et al., 1999). The luciferase values given (RLU=“Relative Light Units” RLU) are averages+SEM of >4 animals.

Measurement of the physical parameters of the PEI/DNA complexes yielded the following particle sizes: PEI(800): 130 nm, PEI(25)/DNA: 180 nm; PEI(22)/DNA: 1-2 μm.

For all the PEI/DNA complexes a strongly positive surface charge was found, which is expressed as a strongly positive zeta potential. The zeta potential of the PEI/DNA complexes was accordingly +30 mV, +35 mV, and +32 mV for PEI(800)/DNA, PEI(25)/DNA, and PEI(22)/DNA complexes. Non-condensed DNA does not form any particles and has a negative zeta potential.

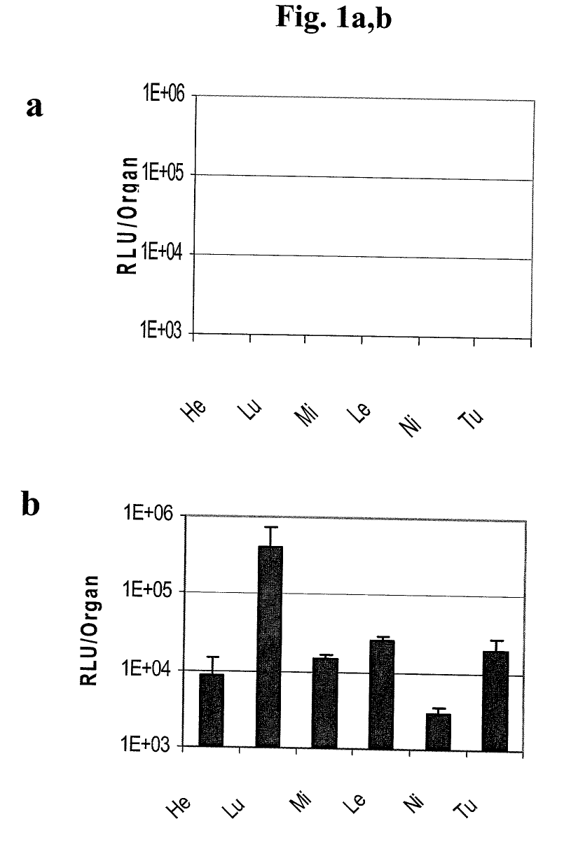

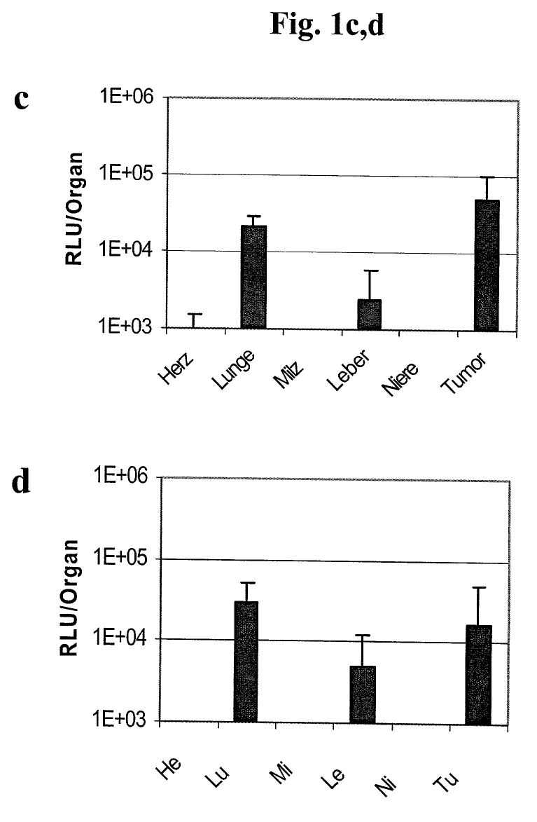

In normal systemic injection the unpackaged, unprotected DNA (FIG. 1a) is quickly broken down in the bloodstream and does not lead to any significant gene expression in the organs under investigation, with the exception of the injection site (not shown in FIG. 1a).

Condensation of the DNA with polycations, shown here for 3 polycations: FIG. 1b) PEI(800), FIG. 1c) PEI25, FIG. 1 d) PEI(22) protects the DNA from immediate breakdown. The resulting complexes have a strongly positive surface charge (positive zeta potential ≧+30 mV).

After systemic administration into the bloodstream significant gene expression values were measured with these polycation/DNA transfection complexes (in each case they contain 50 μg of DNA, volume administered: 250 μl per mouse). The expression pattern is non-specific and highly diversified, with the highest expression values mostly in the lungs; to some extent, gene expression is also measured in the tumour and in other organs such as the heart, liver, kidney and spleen. Severe toxicities were observed in group b) (FIG. 1b), 2 of the 4 animals died shortly after administration with signs of pulmonary embolism. The rest of the animals recovered after clear signs of acute toxicity. Even for the polyethyleneimine/DNA complexes used in this Example the dose which can be administered is limited by acute toxicities after systemic administration.

The data show that the gene expression in the lung and the toxicity are correlated with a positive surface charge (positive zeta potential ≧+30 mV). (In FIG. 1: He=heart, Lu=lung, Mi=spleen, Le=liver, Ni=kidney, Tu=tumour)

### Example 2

Aggregation of Erythrocytes by Positive Surface Charge of Polycation/DNA Complexes

Transfection complexes with the luciferase reporter gene were prepared, as described in Example 1, in 75 mM NaCl, 20 mM HEPES at a DNA concentration of 200 μg/ml.

PEI(800)/DNA complexes (N/P=6) were prepared by rapidly mixing a PEI(800) solution (156 μg PEI(800)/ml in 75 mM NaCl, 20 mM HEPES) and a solution of the DNA (200 μg DNA/ml in 75 mM NaCl, 20 mM HEPES).

PEI(25)/DNA complexes (N/P=6) were prepared by rapidly mixing a PEI(25) solution (156 μg PEI(25)/ml in 75 mM NaCl, 20 mM HEPES) and a solution of the DNA (200 μg DNA/ml in 75 mM NaCl, 20 mM HEPES).

PEI(22)/DNA complexes (N/P=6) were prepared by rapidly mixing a PEI(22) solution (156 μg PEI(22)/ml in 75 mM NaCl, 20 mM HEPES) and a solution of the DNA (200 μg DNA/ml in 75 mM NaCl, 20 mM HEPES).

The transfection complexes were incubated for 20 minutes at ambient temperature after mixing. To ensure isotonicity, glucose was added (to give a final concentration of 2.5 w/v %).

The zeta potential of the PEI/DNA complexes (measured as described in Example 1) was accordingly +30 mV, +35 mV, and +32 mV for PEI(800)/DNA, PEI(25)/DNA, and PEI(22)/DNA complexes.

Fresh blood was taken from A/J mice and 20 μl of heparin was added to prevent clotting. The erythrocytes were washed three times in cold Ringer's solution and seeded onto 6-well cell culture plates.

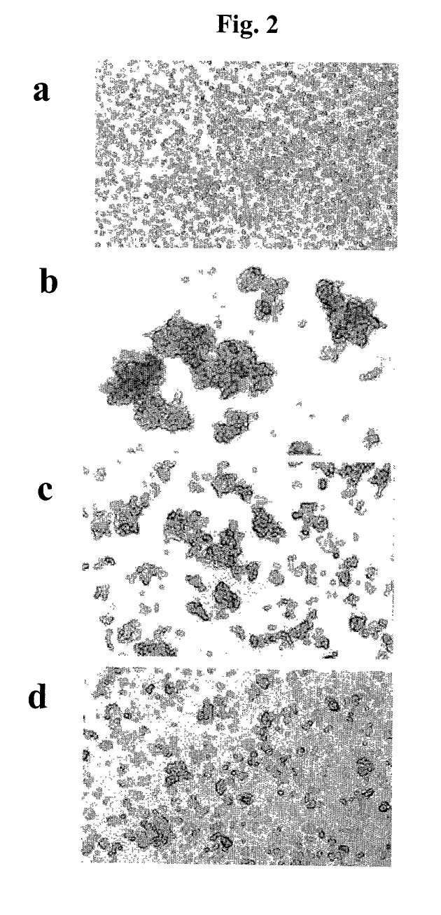

The erythrocytes were combined with the freshly mixed polycation/DNA gene transfer complexes. The final DNA concentration was 17 μg/ml, which corresponds in its order of magnitude to the quantity of DNA administered in vivo (50 μg DNA) per volume of blood of the mouse (2.5-3 ml). The erythrocytes were incubated with the gene transfer complexes for 1 hour at +37° C. and the aggregation of the erythrocytes was evaluated. Untreated erythrocytes are shown as the control (FIG. 2a).

It was found that the incubation of erythrocytes with polycation/DNA complexes (with a positive surface charge, zeta potential: ≧+30 mV) leads to aggregation of erythrocytes (FIG. 2b: PEI(800)/DNA complexes; FIG. 2c: PEI(25)/DNA complexes; FIG. 2d: PEI(22)/DNA complexes. Although clear differences could be observed in the severity of the aggregation between the different polyethyleneimine molecules, the aggregation was significant in every case and illustrates the potential for undesirable effects in vivo. Aggregation of erythrocytes after the administration of transfection complexes into the bloodstream is one of the pathogenic mechanisms of the toxicities described in Example 1, including pulmonary embolism.

### Example 3

Inhibition of Erythrocyte Aggregation by Incorporation of a High Transferrin Content in Polycation/DNA Complexes

a) PEI(25)/DNA transfection complexes (N/P=4.8; N=nitrogen of PEI, P=phosphate of DNA) were prepared analogously to the method described in Example 1 by rapidly mixing a PEI(25) solution (125 μg PEI(25)/ml in 75 mM NaCl, 20 mM HEPES) and a solution of the DNA (200 μg DNA/ml in 75 mM NaCl, 20 mM HEPES).

During the mixing of the complexes, unlike in Example 1 the PEI(25) was partially (e.g. 1/10, ⅕, or ½) or totally replaced by a corresponding amount of transferrin-PEI(25) conjugate (Tf-PEI, with the very high molar ratio of transferrin:PEI of 1:1, described in Materials and Methods). The transfection complexes in which PEI(25) was partially replaced by transferrin-PEI conjugate thus consist of transferrin-PEI(25) conjugate (abbreviated to: Tf-PEI), PEI(25) and DNA, and are hereinafter referred to as “Tf-PEI/PEI(25)/DNA complexes”. The transfection complexes were incubated for 20 minutes at ambient temperature after mixing. To ensure isotonicity, glucose was added (to give a final concentration of 2.5%).

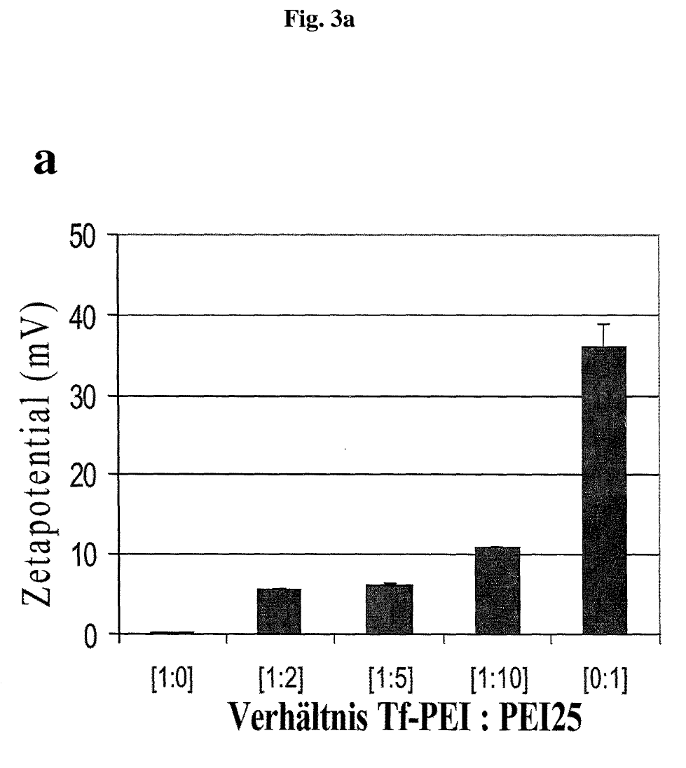

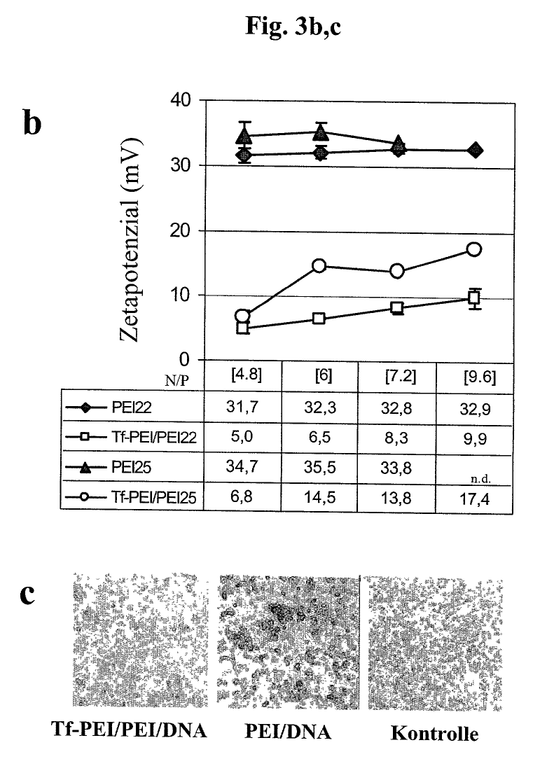

The zeta potential (as an expression of the surface charge) was measured using a Malvern Zetasizer 3000 (as described in Example 1). The effect of incorporating an increasing amount of transferrin conjugate on the zeta potential of the transfection complex was measured (FIG. 3a).

b) Tf-PEI/PEI(25)/DNA transfection complexes (molar ratio of Tf-PEI:PEI=1:3), inter alia N/P=4.8, were prepared by rapidly mixing a polycation solution (consisting of 31 μg/ml Tf-PEI and 94 μg PEI(25)/ml in 75 mM NaCl, 20 mM HEPES) and a solution of the DNA (200 μg DNA/ml in 75 mM NaCl, 20 mM HEPES).

Tf-PEI/PEI(22)/DNA transfection complexes (molar ratio Tf-PEI:PEI=1:3), inter alia N/P=4.8, were prepared by rapidly mixing a polycation solution (consisting of 31 μg/ml Tf-PEI and 94 μg PEI(22)/ml in 75 mM NaCl, 20 mM HEPES) and a solution of the DNA (200 μg DNA/ml in 75 mM NaCl, 20 mM HEPES).

Tf-PEI/PEI(25)/DNA transfection complexes or Tf-PEI/PEI(22)/DNA transfection complexes (with a higher N/P ratio (inter alia N/P=6, 7.2, 9.6) were mixed with correspondingly larger amounts of PEI per constant quantity of DNA. In every case, as in the complexes described above, a quarter of the amount of PEI was replaced with the corresponding amount of Tf-PEI conjugate, i.e. in all these complexes the molar ratio of Tf-PEI:PEI=1:3.

The zeta potential of screened Tf-PEI/PEI(25)/DNA complexes or of Tf-PEI/PEI(22)/DNA complexes and of non-screened PEI(25)/DNA or PEI(22)/DNA complexes was measured over a wide range of N/P ratios (as described in Example 1). FIG. 3b shows the zeta potentials of transfection complexes using PEI25 and PEI22 with and without screening by Tf-PEI.

Of the preferred complexes used in the Examples that follow (N/P=4.8; molar ratio Tf-PEI:PEI=1:3, using PEI25 or PEI22), which were freshly prepared for each experiment, a zeta potential of ≦+10 mV was measured in all the measurements.

c) Influence of the Incorporation of Transferrin in the Complexes on the Aggregation Characteristics of Erythrocytes

Tf-PEI/PEI(25)/DNA transfection complexes (N/P=4.8, Tf-PEI:PEI=1:3) were prepared by rapidly mixing a polycation-solution (consisting of 31 μg/ml Tf-PEI and 94 μg PEI(25)/ml in 75 mM NaCl, 20 mM HEPES) and a solution of the DNA (200 μg DNA/ml in 75 mM NaCl, 20 mM HEPES).

PEI(25)/DNA complexes (N/P=4.8) were prepared by rapidly mixing a PEI(25) solution (125 μg PEI(25)/ml in 75 mM NaCl, 20 mM HEPES) and a solution of the DNA (200 μg DNA/ml in 75 mM NaCl, 20 mM HEPES).

The solutions were made isotonic by the addition of glucose in a final concentration of 2.5%.

Washed fresh erythrocytes were incubated with unscreened PEI25/DNA complexes or with screened Tf-PEI/PEI(25)/DNA complexes (N/P=4.8) for 1 hour at +37° C. and the aggregation of the erythrocytes was evaluated. The results are shown in FIG. 3c, untreated erythrocytes are shown as the control.

It was found that PEI/DNA complexes have a high positive surface charge which is expressed as a strongly positive zeta potential (≧+30 mV).

Incorporation of a large enough amount of transferrin-PEI conjugate (e.g. 10%, 80% or 50%, based on PEI) in the polycation/DNA complex leads to significant screening of the positive surface charge (see FIG. 3a).

By incorporating a large amount of transferrin (¼ based on the molar quantity of total PEI in the complex) in the polycation/DNA complex, the positive surface charge of the PEI/DNA complexes is clearly screened over a wide N/P range. This applies both to Tf-PEI/PEI/DNA complexes containing PEI(25) and to those containing PEI(22) (FIG. 3b).

The screening of the positive surface charge brings about the screening of non-specific interaction, illustrated by the example of the inhibition of erythrocyte aggregation (FIG. 3c).

### Example 4

Systemic Administration of Screened Transferrin-Containing Polycation/DNA Complexes

Transfection complexes screened by transferrin (containing the luciferase reporter gene) were prepared in 75 mM NaCl, 20 mM HEPES at a DNA concentration of 200 μg/ml and incubated for 20 minutes at ambient temperature.

Tf-PEI/PEI(25)/DNA transfection complexes (N/P=4.8, Tf-PEI:PEI=1:3) were prepared by rapidly mixing a polycation-solution (consisting of 31 μg/ml TF-PEI and 94 μg PEI(25)/ml in 75 mM NaCl, 20 mM HEPES) and a solution of the DNA (200 μg DNA/ml in 75 mM NaCl, 20 mM HEPES).

Tf-PEI/PEI(22)/DNA transfection complexes (N/P=4.8, Tf-PEI:PEI=1:3) were prepared by rapidly mixing a polycation-solution (consisting of 31 μg/ml Tf-PEI and 94 μg PEI(22)/ml in 75 mM NaCl, 20 mM HEPES) and a solution of the DNA (200 μg DNA/ml in 75 mM NaCl, 20 mM HEPES).

The transfection complex solutions were made isotonic by the addition of glucose in a final concentration of 2.5%.

The transfection complexes (containing 50 μg DNA/250 μl) were administered systemically through the caudal vein into tumour-bearing A/J mice (neuroblastoma, Neuro2a, growing subcutaneously in the flank) (as described in Example 1).

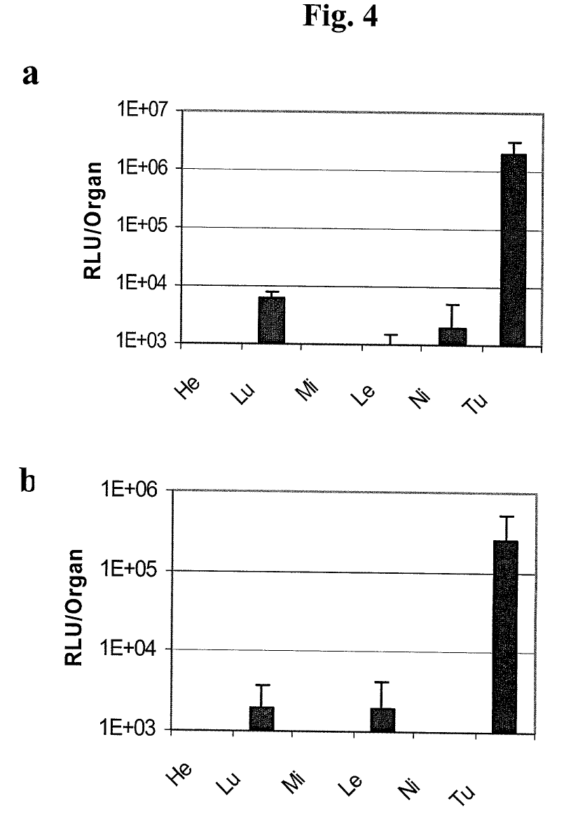

Transfection complexes containing transferrin were tested using PEI25 (FIG. 4a) and PEI22 (FIG. 4b).

The reporter gene expression in the tumour and in the various organs was measured 24 hours after the administration of the transfection complexes by means of a luciferase assay (FIGS. 4a and b). The luciferase values given are the averages ±SEM of 9 animals. (The abbreviations used for the organs have the same meanings as in FIG. 1.)

Systemic administration of transfection complexes in which the charge is screened by transferrin into the mouse's bloodstream led to a preferred reporter gene expression in the tumour, while negligible gene expression was found in the other organs (see logarithmic scale in FIG. 4).

No systemic toxicity occurred after administration of the gene transfer complexes.

### Example 5

Systemic Administration of Screened Transferrin-Containing Polycation/DNA Complexes, Containing TNF-α Plasmid-DNA, in a Neuroblastoma Model

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(25)/DNA transfection complexes (N/P=4.8, Tf-PEI:PEI=1:3) were prepared by rapidly mixing a polycation solution (consisting of 31 μg/ml Tf-PEI and 94 μg PEI(25)/ml in 75 mM NaCl, 20 mM HEPES) and a solution of the DNA (200 μg DNA/ml in 75 mM NaCl, 20 mM HEPES).

The transfection complex solutions were made isotonic by the addition of glucose in a final concentration of 2.5%.

In four applications at intervals of 2 to 3 days, the transfection complexes (each containing 50 μg of DNA/250 μl) were administered systemically through the caudal vein into tumour-bearing A/J mice (10 animals per test group). For this purpose the mice had been injected 8 days previously with 10⁶ tumour cells (neuroblastoma) (Neuro2a ATCC CCL 131) subcutaneously into the flank, and at the time of the first administration of the transfection complexes had a tumour growing subcutaneously with a diameter of 6-8 mm.

The tumour growth was monitored for the next 3 weeks.

Systemic administration of transfection complexes screened by transferrin and containing TNF-α gene into the mouse's bloodstream led to haemorrhagic tumour necrosis in 7 out of 10 of the animals treated. The haemorrhagic necrosis—a TNF-α-specific antitumour effect—was found to be strictly localised in the region of the tumours. No necrosis occurred in normal tissues and there was no systemic TNF-α-mediated toxicity (Example 5a).

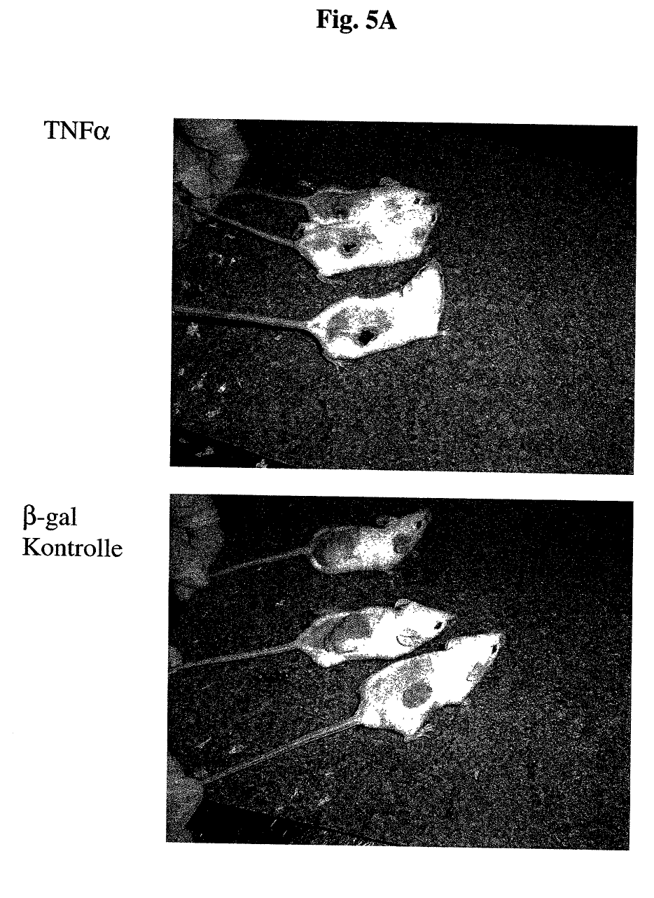

Later on, treatment with the TNF-α-gene-coding transfection complexes resulted in the partial killing off of large areas within the tumours in question and finally resulted in a significant inhibition of the growth of the tumour compared with untreated control animals and also compared with animals that had been treated with the same gene transfer complexes but with different genes (e.g. the β-galactosidase reporter gene) (Experiment according to FIG. 5b).

Once again, no systemic TNF-α-mediated toxicity was found subsequently.

### Example 6

Systemic Administration of Screened Transferrin-Containing Polycation/DNA Complexes, Containing TNF-α Plasmid-DNA in a Fibrosarcoma Model

Transfection complexes screened by transferrin with the gene for TNF-α were prepared in 75 mM NaCl, 20 mM HEPES at a DNA concentration of 200 μg/ml and incubated for 20 minutes at ambient temperature.

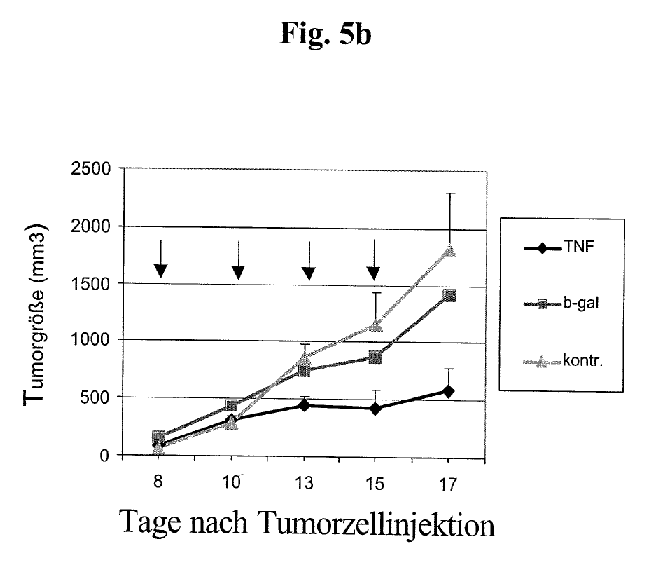

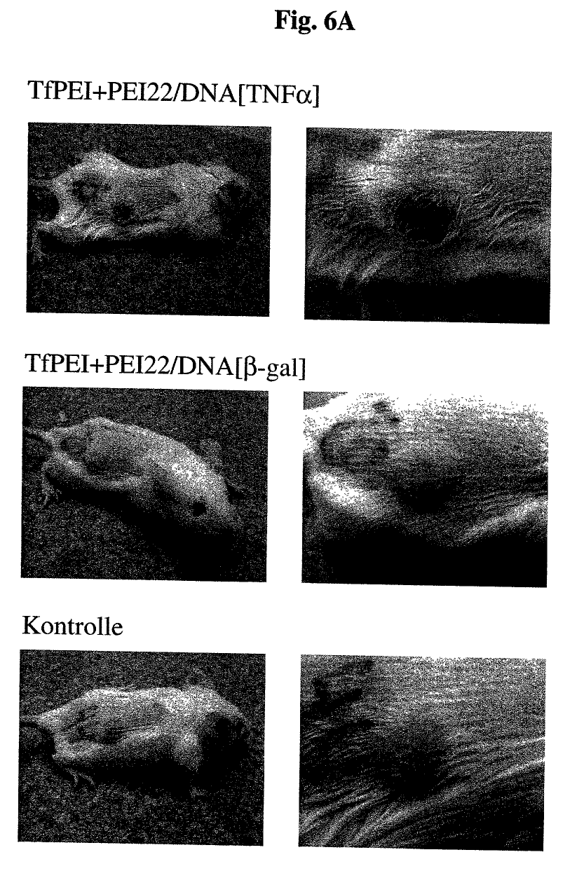

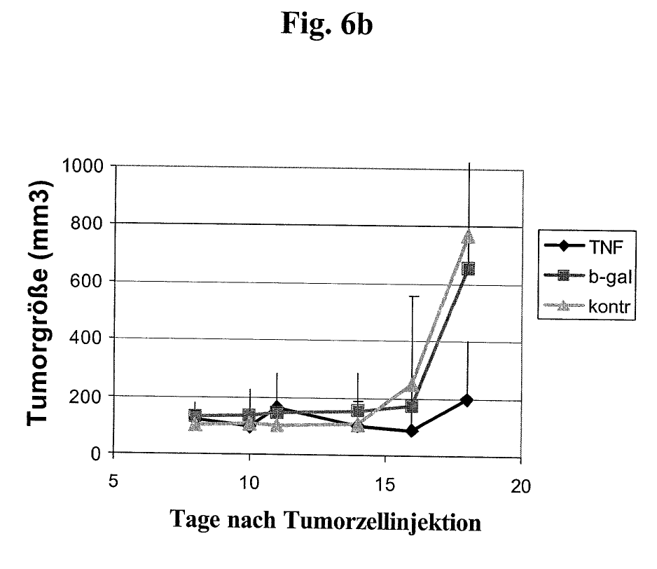

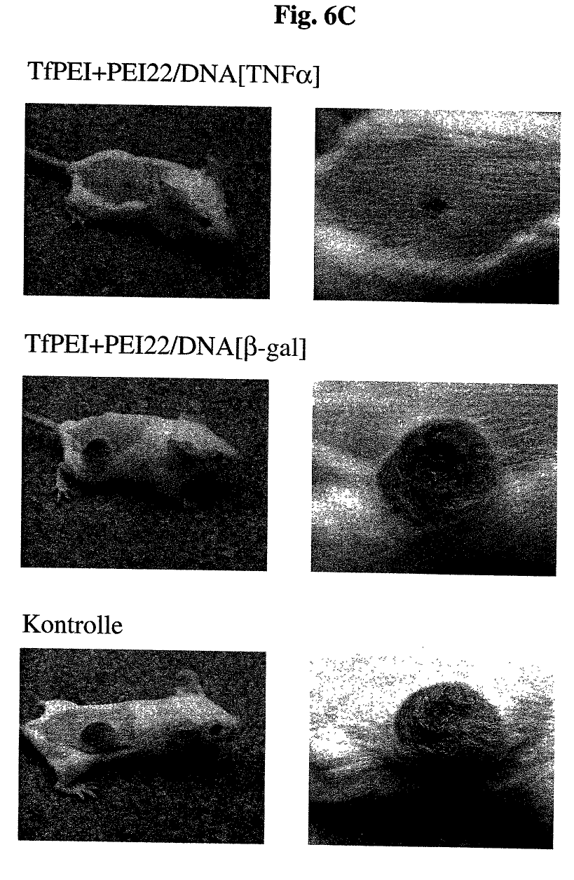

Tf-PEI/PEI(25)/DNA transfection complexes (N/P=4.8, Tf-PEI:PEI=1:3, FIG. 6b) were prepared by rapidly mixing a polycation solution (consisting of 31 μg/ml of Tf-PEI and 94 μg PEI(25)/ml in 75 mM NaCl, 20 mM HEPES) and a solution of the DNA (200 μg DNA/ml in 75 mM NaCl, 20 mM HEPES). The solutions were made isotonic by the addition of glucose in a final concentration of 2.5%.

Tf-PEI/PEI(22)/DNA transfection complexes (N/P=4.8, Tf-PEI:PEI=1:3, FIGS. 6a, c) were prepared by rapidly mixing a polycation solution (consisting of 31 μg/ml Tf-PEI and 94 μg PEI(22)/ml in 20 mM HEPES) and a solution of the DNA (200 μg DNA/ml in 20 mM HEPES). The solutions were made isotonic by the addition of glucose in a final concentration of 5%.

In four applications, the transfection complexes (each containing 50 μg of DNA/250 μl) were administered systemically through the caudal vein into tumour-bearing Balb/c mice having a MethA fibrosarcoma growing subcutaneously in the flank. For this purpose the mice had been injected 8 days previously with 10⁶ MethA tumour cells (MethA fibrosarcoma), and at the time of the first administration of the transfection complexes had a tumour growing subcutaneously with a diameter of 6-8 mm.

The tumour growth was monitored for the next 3 weeks.

Systemic administration of TNF-α-gene-containing transfection complexes in which the charge is screened by transferrin, into the mice's bloodstream led to haemorrhagic tumour necroses in 6 of 10 of the animals treated. The haemorrhagic necrosis—a TNF-α-specific antitumour effect—was found to be strictly localised in the region of the tumours (FIG. 6a). No necrosis occurred in normal tissues and there was no systemic TNF-α-mediated toxicity (Example 6a, b, c).

Later on, treatment with the TNF-α-gene-coding transfection complexes resulted in the partial killing off of large areas within the tumours in question and finally resulted in a significant inhibition of the growth of the tumour compared with untreated control animals and also compared with animals that had been treated with the same gene transfer complexes but with different genes (e.g. the β-galactosidase reporter gene) (FIG. 6b). In 5 out of 10 animals which had been treated with TNF-α gene-coding transfection complexes, total regression of the tumour was observed (FIG. 6c).

Once again, no systemic TNF-α-mediated toxicity was found subsequently.

### Example 7

Systemic administration of screened transferrin-containing polycation/DNA complexes, containing TNF-α plasmid DNA, in a neuroblastoma model leads to a preferred/predominant/expression of TNF-α in the tumour, without any detectable TNF-α serum levels.

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(25)/DNA transfection complexes (N/P=4.8, Tf-PEI:PEI=1:3) were prepared by rapidly mixing a polycation solution (consisting of 31 μg/ml Tf-PEI and 94 μg PEI(25)/ml in 75 mM NaCl, 20 mM HEPES) and a solution of the DNA (200 μg DNA/ml in 75 mM NaCl, 20 mM HEPES). The transfection complex solutions were made isotonic by the addition of glucose in a final concentration of 2.5%.

As a comparison, unscreened PEI(22)/DNA complexes (N/P=7) containing the same TNF-α coding plasmid as the transferrin-screened complexes were prepared by rapidly mixing a PEI(22) solution (182 μg PEI(22)/ml in 20 mM HEPES) and a solution of the DNA (200 μg DNA/ml in 20 mM HEPES).

The solutions were made isotonic by the addition of glucose in a final concentration of 5%.

The transfection complexes (containing 50 μg DNA/250 μl) were systemically administered through the caudal vein into tumour-bearing A/J mice (4 animals per test group). For this purpose the mice had 12 days previously been injected with 10⁶ tumour cells (neuroblastoma) (Neuro2a ATCC CCL 131) subcutaneously into the flank, and at the time of the first administration of the transfection complexes had a tumour growing subcutaneously with a diameter of 10-15 mm.

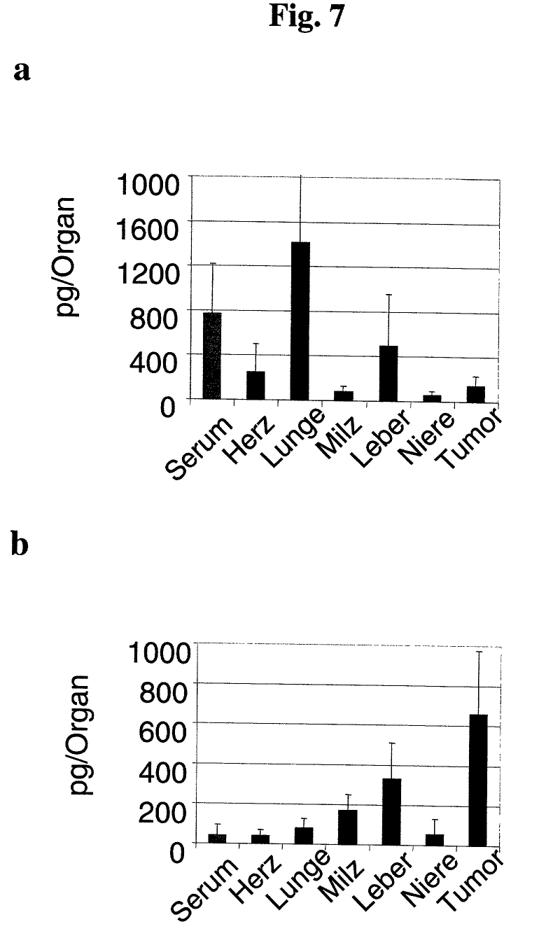

The expression of the gene product, TNF-α, in the tumour and in the various organs, as well as TNF-α serum levels were measured 24 hours after administration of the transfection complexes by means of an ELISA (specific for murine TNF-α) (FIGS. 7a and b). The values given are the averages ±SEM of 4 animals. (The abbreviations used for the organs are the same as in FIG. 1.)

After the systemic administration of unscreened complexes into the bloodstream of mice, a high expression of TNF-α was found in the lung, followed by the liver, heart, tumour and spleen. This non-specific expression in various organs also resulted in significant systemic TNFα levels in the blood serum of the animals (FIG. 7a).

By contrast, the systemic administration of transfection complexes in which the charge is screened by transferrin led to a preferred expression of TNF-α in the tumour, while lesser gene expression was detected in the liver and spleen (see FIG. 7b). Using transferrin-screened transfection complexes no significant TNFα levels were detected in the blood serum of the animals.

### Example 8

Systemic administration of screened transferrin-containing polycation/DNA complexes, containing TNF-α plasmid DNA, in a neuroblastoma model leads to significant inhibition of the tumour growth without any systemic TNF-α-induced toxicity.

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(22)/DNA transfection complexes (N/P=4.8, Tf-PEI:PEI-1:3) were prepared by rapidly mixing a polycation solution (consisting of 31 μg/ml Tf-PEI and 94 μg PEI(22)/ml in 75 mM NaCl, 20 mM HEPES) and a solution of the DNA (200 μg DNA/ml in 75 mM NaCl, 20 mM HEPES). The transfection complex solutions were made isotonic by the addition of glucose in a final concentration of 2.5%.

As a comparison, unscreened PEI(22)/DNA complexes (N/P=7) containing the same TNF-α coding plasmid as the transferrin-screened complexes were prepared by rapidly mixing a PEI(22) solution (182 μg PEI(22)/ml in 20 mM HEPES) and a solution of the DNA (200 μg DNA/ml in 20 mM HEPES).

The solutions were made isotonic by the addition of glucose in a final concentration of 5%.

As controls, corresponding transferrin-screened or unscreened transfection complexes were prepared, analogously to the TNF-α-coding complexes, which contained instead of the TNF-α-coding plasmid the pSP65 plasmid which does not express in mammalian cells.

In eight applications at intervals of 2 to 3 days, the transfection complexes (each containing 50 μg of DNA/250 μl) were administered systemically through the caudal vein into tumour-bearing A/J mice (8 animals per test group). For this purpose the mice had been injected 8 days previously with 10⁶ tumour cells (neuroblastoma) (Neuro2a ATCC CCL 131) subcutaneously into the flank, and at the time of the first administration of the transfection complexes had a tumour 6-8 mm in diameter growing subcutaneously.

The tumour growth was monitored for the next 3 weeks.

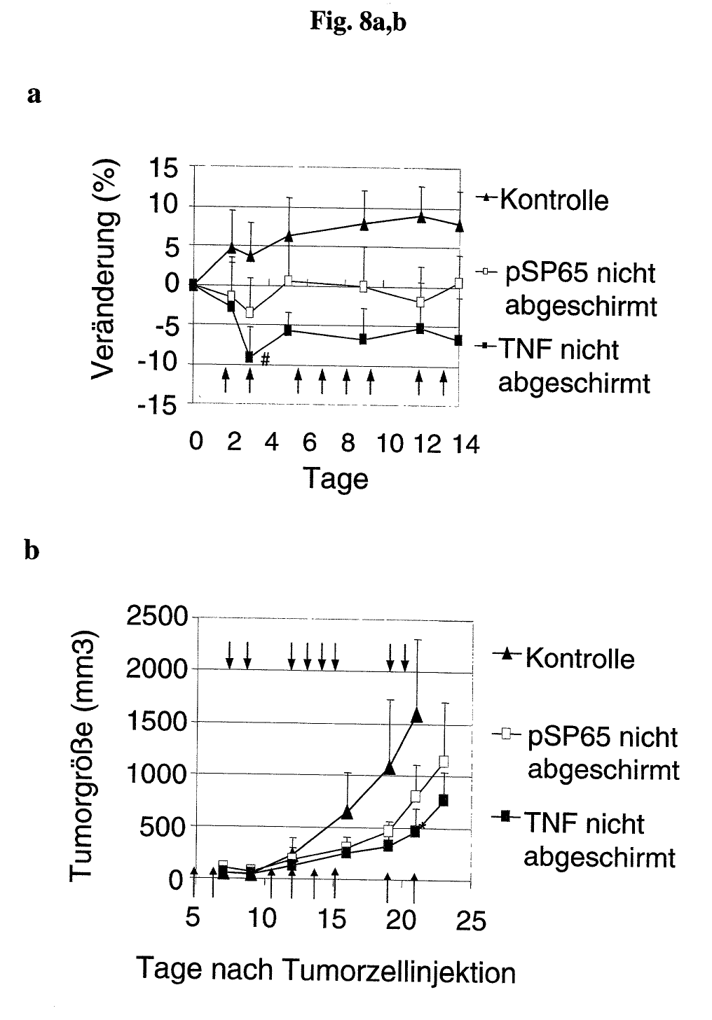

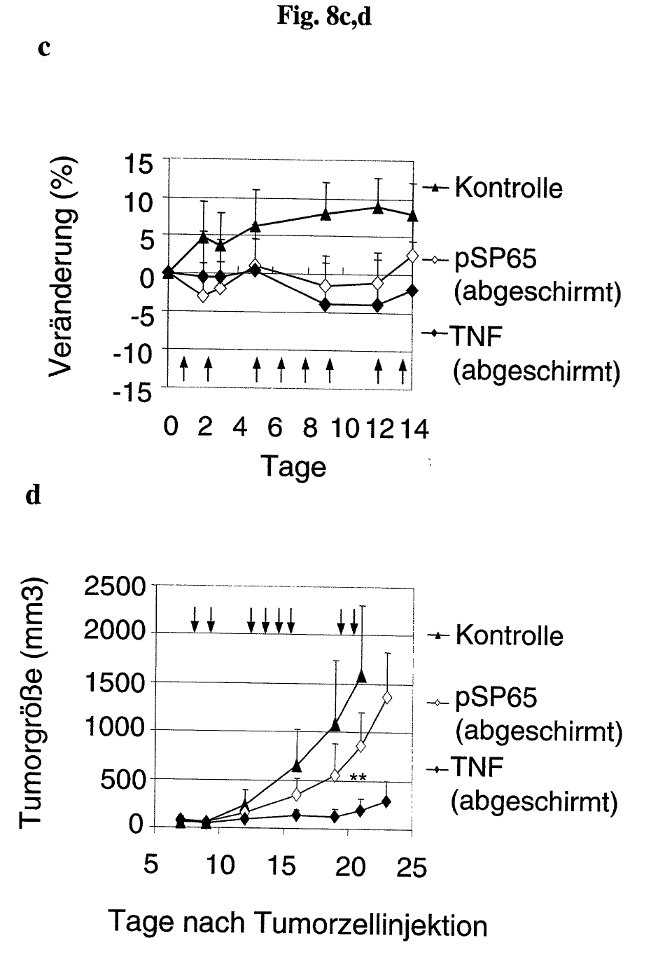

Systemic administration of TNF-α gene-containing transfection complexes screened by transferrin into the mouse's bloodstream led to a significant inhibition of the tumour growth compared with untreated control animals and also compared with animals which had been treated with the same gene transfer complexes, but containing the non-expressing pSP65 plasmid instead of the TNFα coding plasmid (** p<0.01)(experiment according to FIG. 8).

No systemic TNF-α-mediated toxicities were detected after the administration of transferrin-screened transfection complexes.

Systemic administration of unscreened TNF-α gene-containing transfection complexes into the mouse's bloodstream led to a significantly lower inhibition of the tumour growth compared with transferrin-screened complexes (# p<0.05).

Parallel to the tumour growth, the animals' weight was determined as a parameter for any possible influence on the general condition of the animals. Whereas there was no detectable significant weight loss after systemic administration of transferrin-screened transfection complexes, the administration of unscreened transfection complexes with TNF-α led to significant weight losses (p<0.05 vs. untreated control). Unlike the unscreened complexes, transferrin-screened transfection complexes are thus capable of localising the expression of a therapeutic gene (e.g. coding for TNF-α), and hence the activity of the therapeutic protein, TNF-α, to the target site, the tumour, and thereby eliminating the undesirable effects on the normal tissue, of the kind known in systemic TNF-α protein therapy.

### Example 9

Systemic administration of screened transferrin-containing polycation/DNA complexes, containing TNF-α Plasmid-DNA, in a neuroblastoma model leads to haemorrhagic tumour necrosis and significant inhibition of the tumour growth.

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(22)/DNA transfection complexes (N/P=4.8, Tf-PEI:PEI=1:3) were prepared by rapidly mixing a polycation solution (consisting of 31 μg/ml Tf-PEI and 94 μg PEI(22)/ml in 75 mM NaCl, 20 mM HEPES) and a solution of the DNA (200 μg DNA/ml in 75 mM NaCl, 20 mM HEPES).

The transfection complex solutions were made isotonic by the addition of glucose in a final concentration of 2.5%.

In six applications, the transfection complexes (each containing 50 μg of DNA/250 μl) were administered systemically through the caudal vein into the bloodstream of tumour-bearing A/J mice (6 animals per test group). For this purpose the mice had been injected 8 days previously with 10⁶ tumour cells (neuroblastoma) (Neuro2a ATCC CCL 131) subcutaneously into the flank, and at the time of the first administration of the transfection complexes had a tumour 6-8 mm in diameter growing subcutaneously.

The tumour growth was monitored for the next 3 weeks.

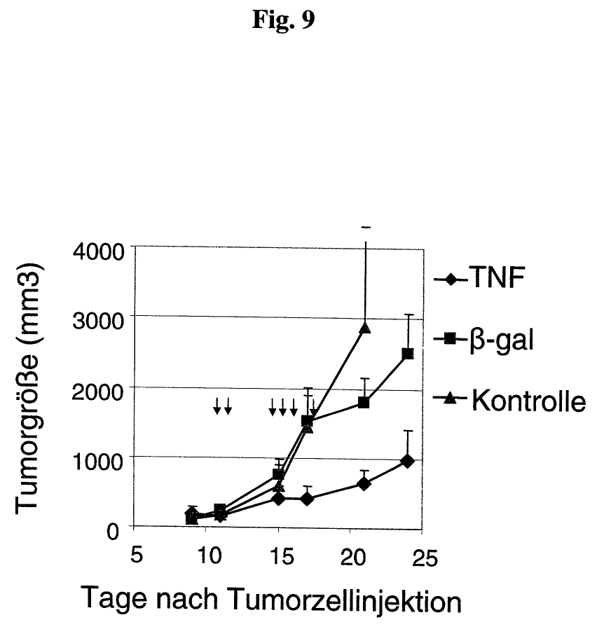

Systemic administration of TNF-α gene-containing transfection complexes screened by transferrin into the mouse's bloodstream led to a significant inhibition of the tumour growth compared with untreated control animals and also compared with animals which had been treated with the same gene transfer complexes, but containing other genes (e.g. the β-galactosidase reporter gene) instead of the TNFα gene (experiment according to FIG. 9).

No systemic TNF-α-mediated toxicities were detected.

Table 1 shows a summary of three independent experiments on the systemic administration of screened transferrin-containing polycation/DNA complexes containing TNF-α plasmid DNA in the neuroblastoma model.

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(25)/DNA or Tf-PEI/PEI(22)/DNA transfection complexes (N/P=4.8, Tf-PEI:PEI=1:3) were prepared by rapidly mixing a polycation solution (consisting of 31 μg/ml Tf-PEI and 94 μg PEI(25)/ml or PEI(22)/ml in 75 mM NaCl, 20 mM HEPES) and a solution of the DNA (200 μg DNA/ml in 75 mM NaCl, 20 mM HEPES).

The transfection complex solutions were made isotonic by the addition of glucose in a final concentration of 2.5%.

As controls, corresponding transferrin-screened transfection complexes were prepared, analogously to the TNF-α-coding complexes, which contained instead of the TNF-α-coding plasmid a therapeutically irrelevant reporter gene (for β-galactosidase) or the pSP65 plasmid which does not express in mammalian cells.

In multiple applications, the transfection complexes (each containing 50 μg of DNA/250 μl) were administered systemically through the caudal vein into the bloodstream of tumour-bearing A/J mice. For this purpose the mice had been injected 8 days previously with 10⁶ tumour cells (neuroblastoma) (Neuro2a ATCC CCL 131) subcutaneously into the flank, and at the time of the first administration of the transfection complexes had a tumour 6-8 mm in diameter growing subcutaneously.

Systemic administration of screened transferrin-containing polycation/DNA complexes, containing TNF-α plasmid DNA, led to haemorrhagic tumour necrosis in 17 out of 20 animals and thus differs significantly (P<0.01) from the untreated control animals or from control animals which had been treated with analogous transfection complexes containing therapeutically irrelevant plasmid DNA (β-galactosidase or pSP65).

### Example 10

Systemic administration of screened transferrin-containing polycation/DNA complexes containing small amounts of TNF-α plasmid DNA in a neuroblastoma model leads to significant inhibition of the tumour growth.

Transfection complexes screened by transferrin, containing 20 μg/250 μl, 10 μg/250 μl or 5 μg/250 μl of a TNF-α coding plasmid, the plasmid used being pGS-muTNF-α with the authentic TNF-α leader sequence, were prepared in 150 mM of NaCl, 20 mM HEPES and incubated for 20 minutes at ambient temperature. Tf-PEI/PEI(22)/DNA transfection complexes (N/P=4.8, Tf-PEI:PEI=1:3) were prepared by rapidly mixing equal volumes of a polycation solution (consisting of 31 μg/ml Tf-PEI and 94 μg PEI(22)/ml in 150 mM NaCl, 20 mM HEPES) and a solution of the DNA (200 μg DNA/ml in 75 mM NaCl, 20 mM HEPES).

In eight applications, the transfection complexes (each containing 20 μg of DNA/250 μl, 10 μg of DNA/250 μl or 5 μg of DNA/250 μl) were administered systemically through the caudal vein into the bloodstream of tumour-bearing A/J mice(12 animals per test group). For this purpose the mice had been injected 8 days previously with 10⁶ tumour cells (neuroblastoma) (Neuro2a ATCC CCL 131) subcutaneously into the flank, and at the time of the first administration of the transfection complexes had a tumour 6-8 mm in diameter growing subcutaneously.

The tumour growth was monitored for the next 3 weeks.

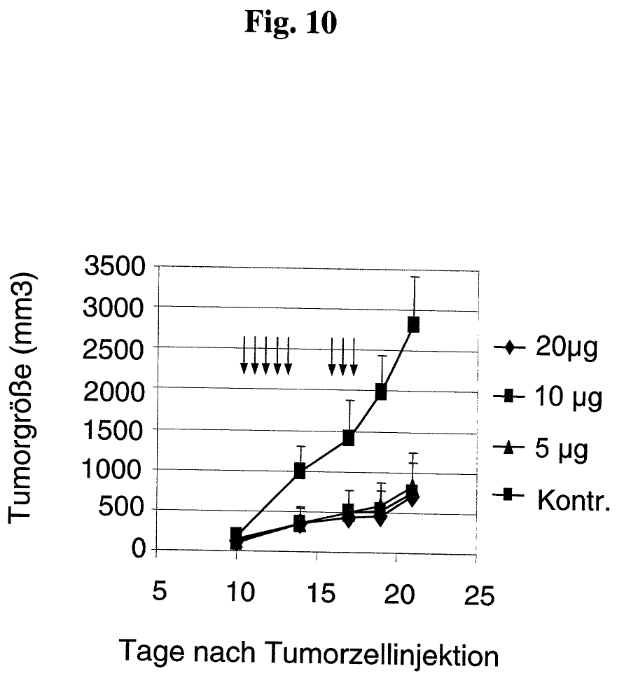

Systemic administration of TNF-α gene-containing transfection complexes screened by transferrin into the mouse's bloodstream led to a significant inhibition of the tumour growth compared with untreated control animals for all the quantities of DNA used (20 μg, 10 μg and 5 μg per mouse) (experiment according to FIG. 10).

No systemic TNF-α-mediated toxicities were found.

### Example 11

Systemic administration of screened transferrin-containing polycation/DNA complexes, containing TNF-α plasmid DNA, in a fibrosarcoma model leads to haemorrhagic tumour necrosis and complete tumour regression.

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(22)/DNA transfection complexes (N/P=4.8, Tf-PEI:PEI=1:3) were prepared by rapidly mixing a polycation solution (consisting of 31 μg/ml Tf-PEI and 94 μg PEI(22)/ml in 75 mM NaCl, 20 mM HEPES) and a solution of the DNA (200 μg DNA/ml in 75 mM NaCl, 20 mM HEPES).

The transfection complex solutions were made isotonic by the addition of glucose in a final concentration of 2.5%.

As controls, corresponding transferrin-screened transfection complexes were prepared, analogously to the TNF-α-coding complexes, which contained instead of the TNF-α-coding plasmid a therapeutically irrelevant reporter gene (for β-galactosidase).

In multiple applications at intervals of 2 to 3 days, the transfection complexes (each containing 50 μg of DNA/250 μl) were administered systemically through the caudal vein into the bloodstream of tumour-bearing Balb/c mice. For this purpose the mice had been injected 8 days previously with 2×10⁶ MethA tumour cells (fibrosarcoma) subcutaneously into the flank, and at the time of the first administration of the transfection complexes had a tumour 6-8 mm in diameter growing subcutaneously.

Table 2 shows a summary of two independent experiments on the systemic administration of screened transferrin-containing polycation/DNA complexes, containing TNF-α plasmid DNA, in the MethA fibrosarcoma model.

Systemic administration of screened transferrin-containing polycation/DNA complexes, containing TNF-α plasmid DNA, led to haemorrhagic tumour necrosis in 11 out of 19 animals and thus differs significantly (** P<0.05) from the untreated control animals or from control animals which had been treated with analogous transfection complexes containing therapeutically irrelevant plasmid DNA coding for β-galactosidase.

Moreover, the administration of screened transferrin-containing polycation/DNA complexes containing TNF-α plasmid DNA led to total tumour regression in 12 out of 19 animals. There is thus a statistically significant difference (# P<0.05) from the untreated control animals.

### Example 12

Systemic administration of screened transferrin-containing polycation/DNA complexes, containing TNF-α plasmid DNA, in the melanoma model M-3 leads to significant inhibition of the tumour growth.

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(22)/DNA transfection complexes (N/P=4.8, Tf-PEI:PEI=1:3) were prepared by rapidly mixing a polycation solution (consisting of 31 μg/ml Tf-PEI and 94 μg PEI(22)/ml in 75 mM NaCl, 20 mM HEPES) and a solution of the DNA (200 μg DNA/ml in 75 mM NaCl, 20 mM HEPES).

The transfection complex solutions were made isotonic by the addition of glucose in a final concentration of 2.5%.

In six administrations the transfection complexes (each containing 50 μg of DNA/250 μl) were administered systemically through the caudal vein into the bloodstream of tumour-bearing DBA/2 mice (10 animals per test group). For this purpose the mice had been injected 8 days previously with 10⁶ M-3 tumour cells (melanoma) subcutaneously into the flank, and at the time of the first administration of the transfection complexes had a tumour 6-8 mm in diameter growing subcutaneously.

The tumour growth was monitored for the next 3 weeks.

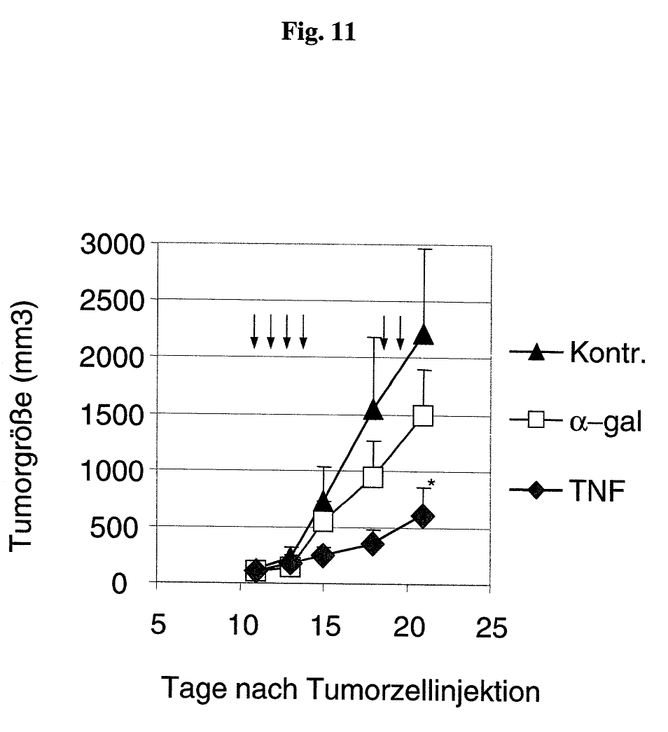

Systemic administration of TNF-α gene-containing transfection complexes screened by transferrin into the mouse's bloodstream led to a significant inhibition of the tumour growth compared with the untreated control animals and also compared with animals which had been treated with the same gene transfer complexes, but with therapeutically irrelevant plasmid DNA (e.g. the β-galactosidase reporter gene) (* p<0.01)(experiment according to FIG. 11).

No systemic TNF-α-mediated toxicities were found.

### Example 13

Systemic administration of screened transferrin-containing polycation/DNA complexes, containing TNF-α plasmid DNA, leads to a significant inhibition of the tumour growth in the melanoma model B16F10.

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(22)/DNA transfection complexes (N/P=4.8, Tf-PEI:PEI=1:3) were prepared by rapidly mixing a polycation solution (consisting of 31 μg/ml Tf-PEI and 94 μg PEI(22)/ml in 75 mM NaCl, 20 mM HEPES) and a solution of the DNA (200 μg DNA/ml in 75 mM NaCl, 20 mM HEPES).

The transfection complex solutions were made isotonic by the addition of glucose in a final concentration of 2.5%.

In six administrations the transfection complexes (each containing 50 μg of DNA/250 μl) were administered systemically through the caudal vein into the bloodstream of tumour-bearing C57B1/6 mice (10 animals per test group). For this purpose the mice had been injected 8 days previously with 10⁶ B15F10 tumour cells (melanoma) subcutaneously into the flank, and at the time of the first administration of the transfection complexes had a tumour 6-8 mm in diameter growing subcutaneously.

The tumour growth was monitored for the next 3 weeks.

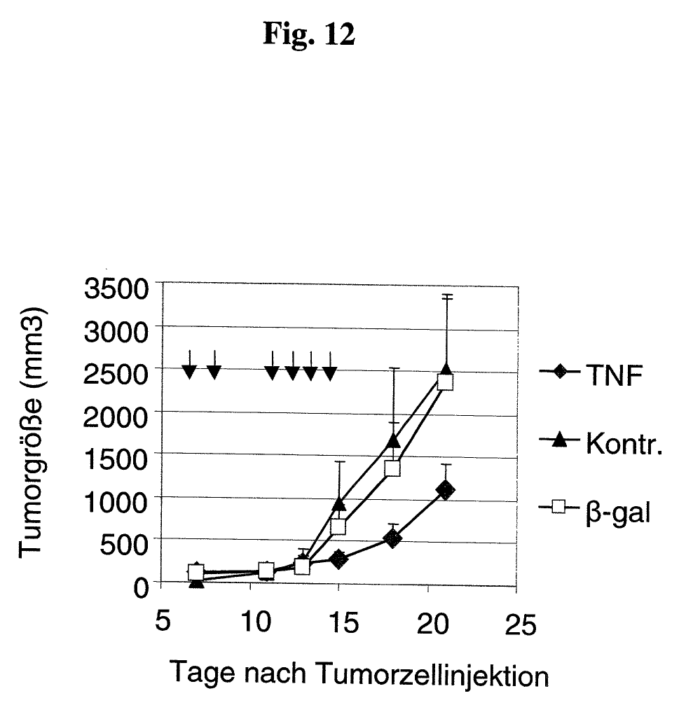

Systemic administration of TNF-α gene-containing transfection complexes screened by transferrin into the mouse's bloodstream led to a slight but significant inhibition of the tumour growth compared with the untreated control animals and also compared with animals which had been treated with the same gene transfer complexes, but with therapeutically irrelevant plasmid DNA (e.g. the β-galactosidase reporter gene) (experiment according to FIG. 12).

No systemic TNF-α-mediated toxicities were found.

### Example 14

Systemic Administration of Screened Transferrin-Containing Polycation/DNA Complexes, Containing TNF-α Plasmid DNA, in Conjunction with a Chemotherapeutic Agent

Transfection complexes screened by transferrin, containing a plasmid coding for TNF-α, the plasmid used being pGS-muTNF-α with the authentic TNF-α leader sequence, were prepared in 75 mM of NaCl, 20 mM HEPES at a DNA concentration of 200 μg/ml and incubated for 20 minutes at ambient temperature. Tf-PEI/PEI(22)/DNA transfection complexes (N/P=4.8, Tf-PEI:PEI=1:3) were prepared by rapidly mixing a polycation solution (consisting of 31 μg/ml Tf-PEI and 94 μg PEI(22)/ml in 75 mM NaCl, 20 mM HEPES) and a solution of the DNA (200 μg DNA/ml in 75 mM NaCl, 20 mM HEPES).

The transfection complex solutions were made isotonic by the addition of glucose in a final concentration of 2.5%.

In seven administrations at intervals of 2 to 3 days the transfection complexes (each containing 50 μg of DNA/250 μl) were administered systemically through the caudal vein into the bloodstream of tumour-bearing C57B1/6 mice (8 animals per test group) as an individual therapy or combined with doxil (first administration: 4.5 mg/kg, all subsequent administrations: 1.5 mg/kg). For this purpose the mice had been injected 8 days previously with 10⁶ B16F10 tumour cells (melanoma) subcutaneously into the flank, and at the time of the first administration of the transfection complexes had a tumour 6-8 mm in diameter growing subcutaneously.

The tumour growth was monitored for the next 3 weeks.

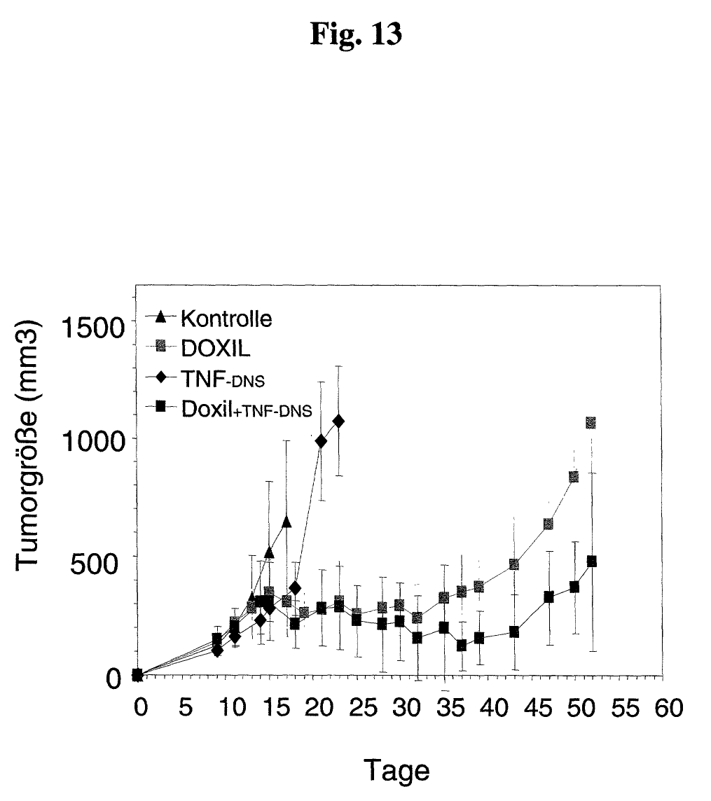

Systemic administration of TNF-α gene-containing transfection complexes screened by transferrin into the mouse's bloodstream led to a significant inhibition of the tumour growth compared with the untreated control animals (experiment according to FIG. 13). However, in combination with doxil, the administration of TNF-α gene-containing transfection complexes screened by transferrin resulted in marked tumour necrosis in 7 out of 8 animals and in a significant inhibition of tumour growth (p<0.01) compared with the untreated control animals.

No systemic TNF-α-mediated toxicities were found.

### Example 15

Systemic Administration of Screened Transferrin-Containing Polycation/DNA Complexes, Containing TNF-α Plasmid DNA, in Conjunction with a Chemotherapeutic Agent

Transfection complexes screened by transferrin, containing a plasmid coding for TNF-α, the plasmid used being pGS-muTNF-α with the authentic TNF-α leader sequence, were prepared in 150 mM of NaCl, 20 mM HEPES at a DNA concentration of 160 μg/ml and incubated for 20 minutes at ambient temperature. Tf-PEI/PEI(22)/DNA transfection complexes (N/P=4.8, Tf-PEI:PEI=1:4) were prepared by rapidly mixing a polycation solution (consisting of 20 μg/ml Tf-PEI and 80 μg PEI(22)/ml in 150 mM NaCl, 20 mM HEPES) and a solution of the DNA (160 μg DNA/ml in 150 mM NaCl, 20 mM HEPES).

In eight administrations at intervals of 2 to 3 days the transfection complexes (each containing 40 μg of DNA/250 μl) were administered systemically through the caudal vein into the bloodstream of tumour-bearing C57B1/6 mice (8 animals per test group) as an individual therapy or on every second application combined with doxil (1.5 mg/kg). For this purpose the mice had been injected 8 days previously with 10⁶ B16F10 tumour cells (melanoma) subcutaneously into the flank, and at the time of the first administration of the transfection complexes had a tumour about 6-8 mm in diameter growing subcutaneously.

The tumour growth was monitored for the next 3 weeks.

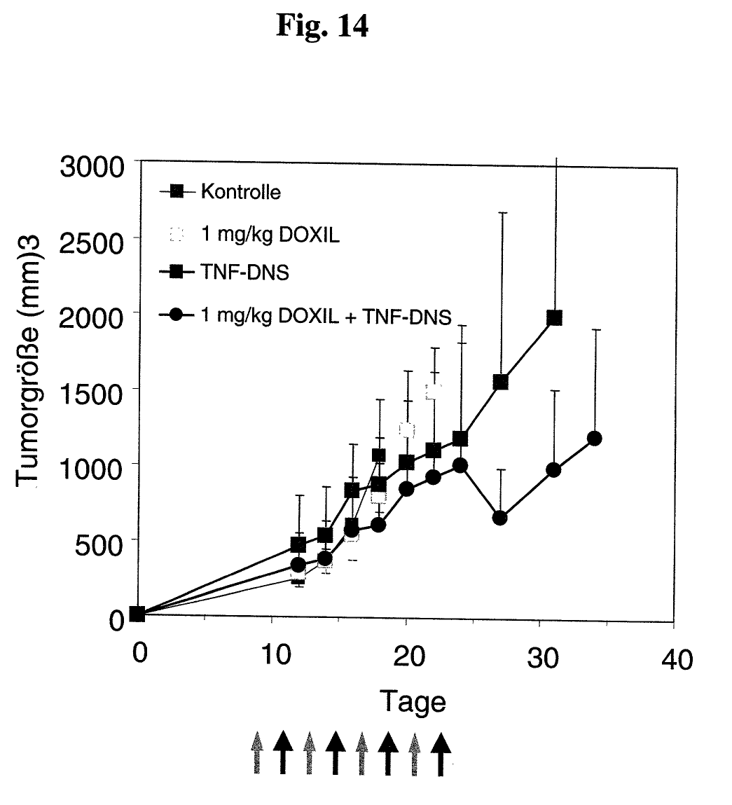

Systemic administration of TNF-α gene-containing transfection complexes screened by transferrin into the mouse's bloodstream led to a significant inhibition of the tumour growth compared with the untreated control animals (experiment according to FIG. 14). However, in combination with doxil, the administration of TNF-α gene-containing transfection complexes screened by transferrin resulted in marked tumour necrosis in all 8 animals and in a significant inhibition of tumour growth (p<0.01) compared with the untreated control animals and also compared with the animals that had been given either TNF-α gene therapy alone or doxil on its own as a monotherapy.

No systemic TNF-α-mediated toxicities were found.

### Example 16

Storage of Screened Transferrin-Containing Polycation/DNA Complexes Over a Lengthy Period

Transfection complexes screened by transferrin, containing a plasmid coding for TNF-α, were prepared in 150 mM NaCl, 20 mM HEPES at a DNA concentration of 80 μg/ml and incubated for 20 minutes at ambient temperature. Tf-PEI/PEI(22)/DNA transfection complexes (N/P=4.8, Tf-PEI:PEI=1:4) were prepared by rapidly mixing a polycation solution (consisting of 10 μg/ml Tf-PEI and 40 μg PEI(22)/ml in 150 mM NaCl, 20 mM HEPES) and a solution of the DNA (80 μg DNA/ml in 150 mM NaCl, 20 mM HEPES). In all 2.5 ml of DNA complexes were prepared.

Directly after the complex formation, the zeta potential and particle size of one aliquot of the freshly formulated DNA complexes were measured using a Zetasizer 3000 (as described in Example 1).

A portion (0.5 ml) of the DNA complexes was further stored at ambient temperature after mixing, while the remainder was flash-frozen in aliquots of 250 μl and stored further at −80° C. Aliquots were taken from both portions, the DNA complexes stored at ambient temperature and those stored at −80° C., at the relevant times and the zeta potential and particle size were measured. For this, the frozen aliquots were rapidly thawed at +37° C.

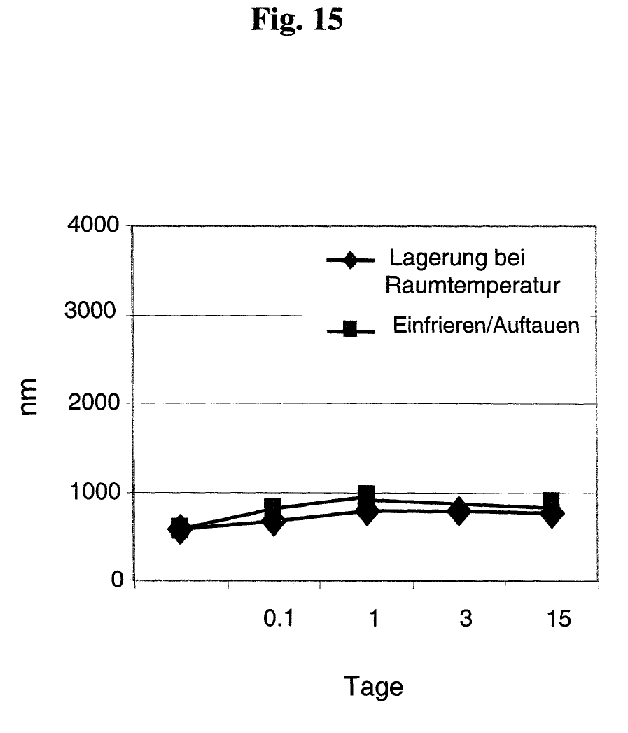

The particle sizes of the DNA complexes measured at the appropriate times are shown in FIG. 15. It can be seen that both the DNA complexes stored at ambient temperature and the frozen DNA complexes are stable for lengthy periods. Similarly, at each time, stable screening of the zeta potential of the complexes was measured (<+10 mV).

TABLE 1
tumour necroses/treated animals

| | Exp 1 | Exp 2 | Exp 3 | Σ |
|---|---|---|---|---|
| TNFα | 6/6 | 4/6 | 7/8 | 17/20* (85%) |
| β-gal | 1/6 | n.d. | n.d. | 1/6 (16%) |
| pSP65 | n.d. | n.d. | 1/8 | 1/8 (12%) |
| control | 0/6 | 0/6 | 1/10 | 1/22 (5%) |

TABLE 2

| | tumour necrosis | | | tumour regression | | |
|---|---|---|---|---|---|---|
| | Exp | Exp | | Exp | Exp | |
| | 1 | 2 | Σ | 1 | 2 | Σ |
| TNFα | 7/10 | 4/9 | 11/19** (58%) | 5/10 | 7/9 | 12/19# (63%) |
| β-gal | 2/10 | 2/9 | 4/19 (21%) | 3/10 | 4/9 | 7/19 (37%) |
| control | 1/10 | 1/9 | 2/19 (11%) | 2/10 | 1/9 | 3/19 (16%) |

### LITERATURE

Abdallah B, Hassan A, Benoist C, Goula D, Behr J P, und Demeneix, B A. A powerful nonviral vector for in vivo gene transfer into the adult mammalian brain: polyethylenimine. Hum Gene Ther 1996; 7: 1947-1954.

Aghi M, Hochberg F, Breakefield X O, Prodrug activation enzymes in cancer gene therapy, J. Gene Med 2000, 2:148-164

Anderson J A, Miller F N, Sims D E, Edwards M J. Tumor necrosis factor causes microvascular protein leakage independently of neutrophils or most cells. J Surg Res 1994; 56(6): 485-490.

Bachwich P R, Chensue S W, Larrick J W, Kunkel S L. Tumor necrosis Factor stimulates Interleukin-1 and prostaglandin E2 production in resting maccrophages. Biochem. Biophys Res Commun 1986; 136:94-101.

Bendtzen K. Interleukin-1, interleukin-6 and tumor necrosis factor in infection, inflammation and immunity. Immunology Letters 1988; 19: 183-192

Beutler B und Cerami A. Cachectin and Tumor Necrosis Factor as two sides of the same biological coin. Nature 1986; 320(17): 584-588.

Beutler B, Milsark I W, Cerami A. Passive immunization against Cachectin/Tumor Necrosis Factor protects mice from lethal effect of endotoxin. Science 1985; 229: 869-871.

Bickels J et al. Isolated limb perfusion with tumor necrosis factor-alpha and melphalan for unresectable bone sarcomas of the lower extremity. Eur J Surg Oncol 1999; 25(5): 509-514.

Blumenthal R. D., et al., (1994), Cancer Res. 54, January 1, 142-151

Boussif O, Lezoulach F, Zanta M A, et al. A versatile vector for gene and oligonucleotide transfer into cells in culture and in vivo-polyethylenimine. Proc Natl Acad Sci USA 1995; 92 (16): 7297-7301.

Bradham C A, Plumpe J, Manns M P, Brenner D A, Trautwein C. Mechanism of hepatic toxicity. I. TNF-induced liver injury. Am J Physiol 1998; 275(3): G387-392.

Brockhous M, Schoenfeld H-J, Schlaeger E-J, Hunziker W, Lesslauer W, Loetscher H. Identification of two types of Tumor Necrosis Factor receptor on human cell lines by monoclonal antibodies. Proc Natl Acad Sci USA 1990; 87: 3127-3131.

Buschle, M. et al. (1995). Receptor mediated gene transfer into human T-lymphocytes via binding of DNA/CD3 antibody particles to the CD3 T cell receptor complex. Hum. Gene Ther. 6:753-761.

Carswell E A, Old L J, Kassel R L, Green S, Fiore N, Williamson B. An endotoxin-induced serum factor that causes necrosis of tumors. Proc Natl Acad Sci USA 1975; 72: 3666-3670.

Cao R., et al., (1999), Proc. Natl. Acad. Sci. USA, Vol. 96, May, 5728-5733.

Collins T, Lapierre L A, Fiers W, Strominger J L, Pober J S. Recombinant human Tumor Necrosis Factor increases nRNA levels and surface expression of HLA-A, B antigens in vascular endothelial cells and dermal fibroblasts in vitro. Proc Natl Acad Sci USA 1986; 83: 446-450.

Cortés M. L., et al., (1998), Gene Therapy 5, 1499-1507.

Creaven P J, Brenner D E, Cowens J W, Huben R P, Wolf R M, Takika H, Arbuck S G et al. A Phase I clinical trial of recombinant human Tumor Necrosis Factor given daily for five days. Cancer Chemother Pharmacol 1989; 23: 186-191.

Dachs, G. U., Patterson, A. V., Firth, J. D., Ratcliffe, P. J., Townsend, K. M. S., Stratford, I. J., und Harris, A. L. (1997). Targeting gene expression to hypoxic tumor cells. Nature Med., 3: 515-520.

Dash P. R., et al., (2000), J. Biol. Chem., Vol. 275, 6, February 11, 3793-3802

Dinarello C A, Cannon J G, Wolff S M, Bernheim H A, Beutler B, Cerami A, et al. Tumor Necrosis Factor (Cachectin) is an endogeneous pyrogen and induces production of Interleukin-1. J Exp Med. 1986; 163: 1433-1500.

Edwards M J, Abney D L, Heniford B T, Miller F N. Passive immunization against tumor necrosis factor inhibits interleukin-2 induced microvascular alterations and reduces toxicity. Surgery 1992; 112(2): 480-486.

Eggermont A M et al. Isolated limb perfusion with tumor necrosis factor and melphalan for limb salvage in 186 patients with locally advanced soft tissue extremity sarcomas. The cumulative multicenter European experience. Ann Surg 1996a; 224(6): 756-764.

Eggermont A M et al. Isolated limb perfusion with high-dose tumor necrosis factor-alpha in combination with interferon-gamma and, elphalan for non-resectable extremity soft tissue sarcomas: a multicenter trial. J Clin Oncol 1996b; 14(10): 2653-2665.

Felgner, J. H., Kumar, R., Sridhar, C. N., Wheeler, C. J., Tsai, Y. J., Border, R., Ramsey, P., Martin, M. und Felgner, P. L. (1994). Enhanced gene delivery and mechanism studies with a novel series of cationic lipid formulations. J. Biol. Chem., 269: 2550-2561.

Ferrero, E., et al. (1996), Tumor necrosis factor alpha-induced vascular leakage involves PECAM1 phosphorylation. Cancer Res 56(14): 3211-3215.

Fritz, J. D., Herweijer, H., Zhang, G., und Wolff, J. A. (1996). Gene transfer into mammalian cells using histone-condensed plasmid DNA. Hum. Gene Ther. 7: 1395-1404.

Gajate, C., et al., (2000), Int. J. Cancer, Vol. 85, 5, 674-682.

Gerlowski L E und Jain R K. Microvascular permeability of normal and neoplastic tissue. Microvasc Res 1986; 31: 288-305.

Granger G A, Williams T W. Lymphocyte cytotoxicity in vitro. Activation and release of a cytotoxic factor. Nature 1968; 218: 1253-1254.

Gray P W, Aggarwal B B, Benton C V, Bringman T S, Henzel W J, Jarret J A, et al. Cloning and expression of cDNS for human Lymphotoxin, a Lymphokine with tumor necrosis activity. Nature 1984; 312: 721-724.

Griscelli, F., et al., (1998), Proc. Natl. Acad. Sci. USA, Vol. 95, May, 6367-6372.

Goula D, Benoist C, Mantero S, Merlo G, Levi G, und Demeneix BA. Polyethylenimine-based intravenous delivery of transgenes to mouse lung. Gene Ther 1998; 5: 1291-1295.

Goula D, Remy J S, Erbacher P, et al. Size, diffusibility and transfection performance of linear PEI/DNS complexes in the mouse central nervous system. Gene Ther 1998; 5: 712-717.

Gregoriadis G. (Ed.). Liposomes as drug carriers: Recent trends and progress. pp. 1-863. John Wiley and Sons, New York, 1988.

Haensler, J. und Szoka, F. C. (1993). Polyamidoamine cascade polymers mediate efficient transfection of cells in culture. Bioconjugate Chem., 4: 372-379.

Haranaka K. Point of view: Tumor Necrosis Factor: how to improve the antitumor activity and decrease accompanying side effects for therapeutic application. J Biol Response Modif 1988; 7: 525-534.

Hirota M, Ogawa M. Shock and its mediators. Nippon Geka Gakkai Zasshi 1999; 100(10): 667-673 (Article in Japanese)

Hohmann H-P, Remy R, Pöschl B, van Loon A P G M. Tumor Necrosis Factor-a and Tumor Necrosis Factor-b bind to the same two types of Tumor Necrosis Factor receptors and maximally activate the transcription factor NF-kB at low receptor occupancy and within minutes after receptor binding. J Biol Chem 1990; 265(25): 15183-15188.

Hong K, Zheng W, Baker A, und Papahadjopoulos D. Stabilization of cationic liposome-plasmid DNS complexes by polyamines and poly(ethylene glycol)-phospholipid conjugates for efficient in vivo gene delivery. FEBS Letters 1996; 400: 233-237.

Hoogenboom, H. R., et al, (1991), Biochim Biophys Acta, June 5, 1096(4):345-54

Jahr J, Grande P O. In vivo effects of tumor necrosis factor-alpha on capillary permeability and vascular tone in a skeletal muscle. Acta Anaesthesiol Scand 1996; 40(2): 256-261.

Jakubowski A A, Casper E S, Gabrilove J L, Templeton M-A, Sherwin S A, Oettgen H F. Phase I Trial of intramuscular administered Tumor Necrosis Factor in patients with advanced cancer. J Clin Oncol 1989; 7(3):298-303.

Jerome,, Vile R G und Hart I R., 1993. In vitro and in vivo targeting of gene expression to melanoma cells. Cancer Res. 53: 962-967.

Jerome, V. und Muller, R., (1998) Hum Gene Ther, December 10; 9(18):2653-9.

Kircheis R, PhD thesis, Humboldt University, Berlin, 1991

Kircheis R, Milleck J, Korobko V G, Shingarova L N, Behnke D, Schmidt H. Biological activity of mutants of human tumour necrosis factor-alpha. Immunology 1992; 76(3): 433-438.

Kircheis R, Milleck J, Korobko V G, Shingarova L N, Schmidt H. Differences in the biological activity of TNFα and TNFβ correlate with their different abilities for binding to the target cells. Eur. Cytokine Network 1992, 3(4): 381-390.

Kircheis R. Kichler A, Wallner G. et al. Coupling of cell-binding ligands to polyethylenimine for targeted gene delivery. Gene Ther 1997; 4: 409-418.

Kircheis, R., Schüller, S., Brunner, S., Ogris, M., Heider, K.-H., Zauner, W., und Wagner, E. (1999) Polycation-based DNS complexes for tumor-targeted gene delivery in vivo. J. Gene Medicine 1, 111-120.

Klebanoff S J, Vadas M A, Harlan J M, Sparks L H, Gamble J R, Agosti J M, Waltersdorph A M. Stimulation of neutrophiles by Tumor Necrosis Factor. J Immunol 1986; 136: 4220-4225.

Kline J A, Thornton L R, Lopaschuk G D, Barbee R W, Watts J A. Heart function after severe hemorrhagic shock. Shock 1999; 12(6): 454-461.

Kramer S M, Carver M E, Apperson S M. Comparison of TNF-α and TNF-β cytolytic biological activities in a serum-free bioassay. Lymphokine Res 1986; 5: S139-S143.

Kukowska-Latallo, J. F., Bielinska, A. U., Johnson, J., Spindler, R., Tomalia, D. A., und Baker, J. R. (1996). Efficient transfer of genetic material into mammalian cells using Starburst polyamidoamine dendrimers. Proc. Natl. Acad. Sci. USA, 93: 4897-4902.

Kunstle G, Hentze H, Germann P G, Tiegs G. Meergans T, Wendel A. Concanavalin A hepatotoxicity in mice: tumor necrosis factor-mediated organ failure independent of caspase-3-like protease activation. Hepatology 1999; 30(5): 1241-1251.

Lee E R, Marshall J, Siegel C S, et al. Detailed analysis of structure and formulations of cationic lipids for efficient gene transfer to the lung. Hum Gene Ther 1996; 7: 1701-1707.

Lenk H, Tanneberger S, Müller U, Ebert J, Shiga T, Phase II clinical trial of high-dose recombinant human Tumor Necrosis Factor. Cancer Chemother Pharmacol. 1989; 24: 391-392.

Li, S und Huang L. In vivo gene transfer via intravenous administration of cationic lipid-protamine-DNS (LPD) complexes. Gene Ther 1997; 4: 891-900.

Lienard D et al. Isolated limb perfusion with tumour necrosis factor-alpha and melphalan with or without interferon-gamma for the treatment of in-transit melanoma metastases: a multicentre randomized phase II study. Melanoma Res 1999; 9(5): 491-502.

Liu F, Qi H, Huang L, und Liu D. Factors controlling the efficiency of cationic lipid-mediated transfection in vivo via intravenous administration. Gene Ther 1997; 4: 517-523.

Liu Y., Mounkes, L. C., Liggitt, H. D., Brown, C. S., Solodin, I., Heath, T. D., und Debs, R. J. 1997. Factors influencing the efficiency of cationic liposome-mediated intravenous gene delivery. Nature Biotechnology 15: 167-173.

Mahato R I, Kawabata K, Nomura T, Takakura Y, and Hashida M. Physicochemical and pharmacokinetic characteristics of plasmid DNS/cationic liposome complexes. J Pharmacol Sci 1995; 84: 1267-1271.

Männel D N, Moore R N, Mergenhagen S E. Macrophages as a source of tumoricidal activity (Tumor Necrosis Factor). Infect Immunity 1980; 30: 523-530.

Massuda, E. S., et al., (1997), Proc. Natl. Acad. Sci. USA, Vol. 94, Dec., 14701-14706.

Mizuguchi, H., et al., (1998), Cancer Res 58, Dec. 15, 5725-5730.

Mori M, et al. High levels of cytokine-producing cells in the lung tissues of patients with fatal hantovirus pulmonary syndrome. J Infect Dis 1999; 179 (2): 295-302.

Murphey E D, Traber D L. Pretreatment with tumor necrosis factor-alpha attenuates arterial hypotension and mortality induced by endotoxin in pigs. Crit Care Med 2000; 28(6): 2015-2021.

Müller R H, 1996, ZetaPotenzial und Partikelladung in der Laborpraxis, Wissenschaftliche Verlagsgesellschaft W V G Stuttgart

Nagamachi, Y., et al., (1999), Cancer Gene Ther. Vol. 6, No. 6, 546-553.

Natanson C, Eichenholz P W, Danner R L, Eichacker P Q, Hoffman W D, Kuo G C et al. Endotoxin and Tumor Necrosis Factor challenges in dogs simulate the cardiovascular profile of human septic schock. J Exp Med 1989; 169: 823-832.

Ogris M, Brunner S, Schüller S, Kircheis R, und Wagner E. PEGylated DNS/Transferrin-PEI complexes: Reduced interaction with blood components, extended circulation in blood and potential for systemic gene delivery. Gene Ther 1999, 6: 595-605.

Ogris M, Steinlein P, Kursa M, Mechtler K, Kircheis R, und Wagner E. The size of DNS/transferrin-PEI complexes is an important factor for gene expression in cultured cells. Gene Ther 1998; 5: 1425-1433.

Old L J. Tumor Necrosis Factor (TNF). Science 1985; 230: 630-632.

Olieman A F, et al. Hyperthermic isolated limb perfusion with tumor necrosis factor alpha. interferon gamma, and melphalan for locally advanced nonmelanoma skin tumors of the extremities: a multicenter study. Arch Surg 1999; 134(3): 303-307.

Otto U, Schneider A W, Conrad S, Kempeni J, Klosterhalfen H. Efficiency and toxicity of Tumor Necrosis Factor-a and a-2a-Interferon in metastatic renal cell carcinoma: Results of a Phase II study. 3^{rd} Intern Conference on Tumor Necrosis Factor and Related Cytokines. Chiba, Japan, 1990, Abstracts, 194.

O'Reily, M. S., et al., (1997), Cell, Vol. 88, January 24, 277-285.

Papahadjopoulos D, Allen T M, Gabizon A, et al. Sterically stabilized liposomes: Improvements in pharmacokinetics and antitumor therapeutic efficacy. Proc Natl Acad Sci USA 1991; 88: 11460-11464.

Pfreundschuh M G, Steinmetz H T, Tüschen R, Schenk V, Diehl V, Schaadt M. Phase I study of intratumoral application of recombinant human Tumor Necrosis Factor. Eur J Cancer Clin Oncol 1989; 25(2): 379-388.

Plank C, Mechtler K, Szoka F, und Wagner E. Activation of the complement system by synthetic DNS complexes: A potential barrier for intravenous gene delivery. Hum Gene Ther 1996; 7, 1437-1446.

Pober J S. TNF as an activator of vascular endothelium. 22^{nd} Forum in Immunology, Paris, 1988: The multiple roles of Tumor Necrosis Factor. Ann. Inst. Pasteur/Immunol. 1988; 139: 317-323.

Quinn J V, Slotman G J. Platelet-activating factor and arachidonic acid metabolites medoate tumor necrosis factor and eicosanoid kinetics and cardiopulmonary dysfunction during bacteremic shock. Crit Care Med 1999; 27(11): 2485-2494.

Renard N, Lienard D, Lespagnard L, Eggermont A, Heimann R, Lejeune F. Early endothelium activation and polymorphonuclear cell invasion precede specific necrosis of human melanoma and sarcoma treated by intravascular high-dose tumour necrosis factor alpha (rTNF alpha). Int J Cancer 1994; 57(5): 656-663.

Renard N, et al. VWF release and platelet aggregation in human melanoma after perfusion with TNF alpha. J Pathol 1995; 176(3): 279-287.

Rosenthal, D. I., et al., (1999), Clin. Cancer Res., Vol. 5, April, 739-745.

Scheurich P, Thoma B, Ücer U, Pfizenmaier K. Immunoregulatory activity of recombinant human Tumor Necrosis Factor (TNF)-alpha: Induction of TNF receptors on human T cells and TNF-alpha mediated enhancement of T cell responses. J. Immunol. 1987; 138: 1786-1790.

Schmidt, W. et al. (1995). Cancer vaccines: The interleukin 2 dosage effect. PNAS, 92: 4711-4714.

Schwartz, B., Benoist, C., Abdallah, B., Scherman, D., Behr, J. P., und Demeneix, B. A. (1995). Lipospermine-based gene transfer into newborn mouse brain is optimized by a low lipospermine/DNA charge ratio. Hum. Gene Ther., 6: 1515-1524.

Sherman M L, Spriggs D R, Arthur K A, Imamura A, Frei E, Kufe D W. Recombinant human Tumor Necrosis Factor administered as a five-day contimuous infusion in cancer patients: Phase I toxicity and effects in lipid metabolism. J Clin Oncol 1988; 6(2): 344-350.

Song Y K, Liu F, Chu S, und Liu D. Characterization of cationic liposome-mediated gene transfer in vivo by intravenous administration. Hum Gene Ther 1997; 8: 1585-1594.

Stam T C, Swaak A J, de Vries M R, ten Hagen T L, Eggermont A M. Systemic toxicity and cytokine/acute phase protein levels in patients after isolated limb perfusion with tumor necrosis factor-alpha complicated by high leakage. Ann Surg Oncol 2000; 7(4): 268-275.

Sugarman B J, Aggarwal B B, Hass P E, Figari I S, Palladino M A, Shepard H M. Recombinat Tumor Necrosis Factor-a: Effect on proliferation of normal and transfromed cells in vitro. Science 1985; 230: 943-945.

Tang, M. X., Redemann, C. T., und Szoka, F. C. Jr. (1996). In vitro gene delivery by degraded polyamidoamine dendrimers. Bioconj. Chem., 7: 703-714.

Templeton N S, Lasic D D, Frederik P M, Strey H H, Robert D D, und Pavlakis G N. Improved DNS: liposome complexes for increased systemic delivery and gene expression. Nature Biotechnology 1997; 15: 647-652.

Ter H G J. TNF-alpha-induced selective cerebral endothelial leakage and increased mortality risk in postmyocardial infarction depression. Am J Physiol 1998; 275(5): H1910-1911.

Thurston, G., McLean, J. W., Rizen, M., Baluk, P., Haskell, A., Murphy, T. J., Hanahan, D., und McDonald, D. M. (1998). Cationic liposomes target angiogenic endothelial cells in tumors and chronic inflammation in mice. J. Clin. Invest., 101: 1401-1413.

Utsumi, T. et al. 1995. Human Pro-Tumor Necrosis Factor: Molecular Determinants of membrane Translocation, Sorting and Maturation. Mol.Cell.Biol. 15:6398-6405

Vacik, J., Dean, B. S., Zimmer, W. E., und Dean, D. A. (1999). Cell-specific nuclear import of plasmid DNA. Gene Ther., 6: 1006-1014.

Van de Wetering, P., Cherng, J. Y., Talsma, H., Crommelin, D. J., und Hennink, W. E. (1998). 2-(Dimethylamino)ethyl methacrylate based (co)polymers as gene transfer agents. J. Controlled Release, 53: 145-153.

Van de Wiel, Bloksma N, Kuper C F, Hofhuis F M, Willers J M. Macroscopic and microscopic early effects of Tumor Necrosis Factor on murine Meth A sarcoma, and relation to curative activity. J Pathol 1989; 157: 65-73.

Van der Veen A H, Seynhaeve A L, Breurs J, Nooijen P T, Marquet R L, Eggermont A M. In vivo isolated kidney perfusion with tumour necrosis factor alpha (TNF-alpha) in tumor-bearing rats. Br J Cancer 1999; 79(3-4): 433-439.

Vile, R. G. und Hart, I. R. (1993). In vitro and in vivo targeting of gene expression to melanoma cells. Cancer Res., 53: 962-967.

von Heijne, G., (1983). Patterns of Amino Acids near Signal-Sequence Cleavage Sites. Eur.J.Biochem. 133: 17-21.

Vrouenraets B C, Kroon B B, Ogilvie A C, van Geel A N, Nieweg O E, Swaak A J, Eggermont A M. Absence of severe systemic toxicity after leakage-controlled isolated limb perfusion with tumor necrosis factor-alpha and melphalan. Ann Surg Oncol 6(4): 405-412.

Wagner, E., Zenke, M., Cotten, M., Beug, H., und Birnstiel, M. L. (1990). Transferrin-polycation conjugates as carriers for DNA uptake into cells. Proc. Natl. Acad. Sci. USA, 87: 3410-3414.

Wagner, E., Cotten, M., Foisner, R., Birnstiel, M. L. (1991). Transferrin-polycation-DNA complexes: The effect of polycations on the structure of the complex and DNA delivery to cells. Proc. Natl. Acad. Sci. USA, 88: 4255-4259.

Wagner E, Curiel D, Cotten M. Delivery of drugs, proteins and genes into cells using transferrin as a ligand for receptor mediated endocytosis. Adv Drug Del Rev 1994; 14:113-135.

Watanabe N, Niitsu Y, Umeno H, Kuriyama H, Neda H, Yamauchi N, Maeda M, Urushizaki I. Toxic effect of Tumor Necrosis Factor on tumor vasculature in mice. Cancer Res 1988; 48: 2179-2183.

Westermann S, Vollmar B, Thorlacius H, Menger M D. Surface cooling inhibits tumor necrosis factor-alpha induced microvascular perfusion failure, leukocyte adhesion, and apoptosis in the striated muscle. Surgery 1999; 126(5): 881-889.

Xu, L., et al., (1999), Tumor Targeting, 4, 92-104.

Yi E S, Ulich T R. Endotoxin, interleukin-1, and tumor necrosis factor cause neutrophil-dependent microvascular leakage in postcapillary venules. Am J Pathol 1992; 140(3): 659-663.

Zanta, M. A., Belguise-Valladier, P., Behr, J. P. (1999). Gene delivery: A single nuclear localization signal peptide is sufficient to carry DNA to the cell nucleus. Proc. Natl. Acad. Sci. USA, 96: 91-96.

1. Complex for the treatment of tumour diseases by systemic administration of DNA, containing, in expressible form, one or more DNA molecules, coding for one or more therapeutically active proteins with a cytotoxic activity and a polycation which condenses the DNA and is wholly or partly conjugated with transferrin, characterised in that the complex has a surface charge which corresponds to a zeta potential of ≦+15 mV, obtained by measuring in aqueous solution at a concentration of ≧10 mM NaCl, more than 50% of the screening of the positive charges in the complex being effected by transferrin.

2. Complex according to claim 1, characterised in that the zeta potential is +10 mV to −10 mV.

3. Complex according to claim 1, characterised in that the zeta potential is +5 mV to −5 mV.

4. Complex according to claim 1, characterised in that the DNA codes for TNF-α and/or for TNF-β and/or IL-1 and/or IL-6.

5. Complex according to claim 4, characterised in that the DNA codes for TNF-α.

6. Complex according to one of the preceding claims, characterised in that preceding the DNA sequence is a secretory leader sequence.

7. Complex according to claim 5 and 6, characterised in that the leader sequence is the TNF-α Type II leader sequence.

8. Complex according to claim 6, characterised in that the leader sequence is a Type I immunoglobulin leader sequence.

9. Complex according to claim 1, characterised in that the DNA codes for IFN-α or for IFN-γ.

10. Complex according to claim 1, characterised in that the DNA codes for a toxin.

11. Complex according to claim 1, characterised in that the DNA codes for a suicide gene.

12. Complex according to claim 11, characterised in that the suicide gene is the Herpes Simplex Thymidine kinase gene.

13. Complex according to claim 1, characterised in that the polycation conjugated with transferrin is selected from among the polyethyleneimines, homologous polycationic polypeptides, histones, spermines, spermidines, cationic lipids and dendrimers.

14. Complex according to claim 13, characterised in that the polycation is a linear or branched polyethyleneimine with an average molecular weight of about 2000 D and 800,000 D.

15. Complex according to claim 13, characterised in that the homologous polycationic polypeptide is polylysine.

16. Complex according to claim 1, characterised in that the N/P ratio is about 0.5 to about 100.

17. Complex according to claim 16, characterised in that the N/P ratio is about 2 to about 20.

18. Complex according to claim 17, characterised in that the N/P ratio is about 4 to about 10.

19. Complex according to claim 1, characterised in that the ratio of transferrin:polycation (w/w) is about 3:1 to about 1:4.

20. Complex according to claim 1, characterised in that the complex contains a proportion of non-transferrin-conjugated polycation, the molar ratio of transferrin-conjugated polycation:non-conjugated polycation being about 1:0 to about 1:20.

21. Complex according to claim 1 or 20, characterised in that the transferrin-conjugated or the non-conjugated polycation is conjugated with a hydrophilic polymer.

22. Complex according to claim 21, characterised in that the hydrophilic polymer is a polyethyleneglycol.

23. Complex according to claim 21 or 22, characterised in that at most 30% of the screening of the positive charges is effected by the hydrophilic polymer.

24. Pharmaceutical composition, containing as active ingredient one or more of the complexes defined in one of claims 1 to 23.

25. Pharmaceutical composition according to claim 24, further containing a chemotherapeutic agent.

26. Pharmaceutical composition according to claim 25, characterised in that the chemotherapeutic agent is selected from among doxorubicin, taxol, 5-fluorouracil, cisplatin and vinblastin.

27. Use of a complex according to one of claims 1 to 23 for administration in the treatment of cancer in conjunction with a preceding, simultaneous or subsequent administration of a chemotherapeutic agent.

28. Use of a complex according to claim 27, wherein the chemotherapeutic agent is selected from among doxorubicin, taxol, 5-fluorouracil, cisplatin and vinblastin.
